(19) United States
(12) Patent Application Publication
(10) Pub. No.: US 2022/0323761 A1
(43) Pub. Date: Oct. 13, 2022

(54) METHOD FOR INDUCING HIBERNATION-LIKE STATE AND DEVICE SAME

(71) Applicant: University of Tsukuba, Tsukuba-shi (JP)

(72) Inventors: Takeshi SAKURAI, Ibaraki (JP); Toru TAKAHASHI, Ibaraki (JP); Genshiro SUNAGAWA, Saitama (JP)

(73) Assignee: University of Tsukuba, Tsukuba-shi (JP)

(21) Appl. No.: 17/764,839

(22) PCT Filed: Sep. 30, 2020
(86) PCT No.: PCT/JP2020/037268

§ 371 Date: Mar. 29, 2022

(30) Foreign Application Priority Data
Sep. 30, 2019 (JP) 2019-178611

(51) Int. Cl. A61N 1/36; A61N 1/05; A61M 21/02; G01N 33/50; A61K 49/00

(52) U.S. Cl. CPC A61N 1/36078; A61N 1/36121; A61N 1/36135; A61N 1/0529; A61M 21/02; G01N 33/5058; A61K 49/0004; A61M 2230/50; A61M 2230/43; A61M 2021/0077

(57) ABSTRACT

A method for inducing a hibernation-like state and a device for the same is described. The method is a chemical and physical method for reducing, in a subject, a theoretical set-point temperature of a body temperature and/or a feedback gain of heat production, or for inducing a hibernation-like state in the subject, the method including applying an excitatory stimulus to pyroglutamylated RFamide peptide (QRFP)-producing neurons. A device used to implement the method is also described.

### TECHNICAL FIELD

The present invention provides a method for inducing a hibernation-like state and a device for the same.

### BACKGROUND ART

Homeothermic animals, birds, and mammals consume most of their body energy for heat production in order to maintain an internal body temperature (T_{B}) within a narrow range that is higher than the ambient temperature (T_{A}). However, some mammals actively lower their body temperature and enter into a state known as hibernation in order to survive food scarcity during the winter. The animals then return to a normal state without any obvious tissue damage^{1,2}. Mice do not hibernate, but they exhibit a short-term hypometabolic state known as daily torpor if a benefit can be obtained from a reduction in basal metabolism. In both hibernation and daily torpor, the reduction in energy consumption is primarily achieved by reducing the body temperature, and the reduction in body temperature is influenced by two main factors, namely, the theoretical set-point temperature (TR) and the negative feedback gain (H) of heat production. In mice undergoing daily torpor, TR remains close to normal, but H is reduced to nearly one-tenth of normal, resulting in a TB that is significantly lower than TR³. In contrast, in hibernation, TR and H are both significantly reduced, and as a result, a hypometabolic state that is more efficient than daily torpor and can respond to changes in outside air temperature can be maintained^{4,5}. It has been confirmed in numerous experiments that this type of aggressive metabolic reduction is regulated by the central nervous system⁶. However, the neural mechanism remains entirely unclear. Elucidation of the mechanisms of daily torpor and/or hibernation is a step that is necessary for developing a method for artificially inducing an artificial hibernation-like metabolically reduced state in non-hibernating animals including humans^{1,7}. Furthermore, such developments would also be beneficial in long-range space exploration in the future. Here, it was discovered that excitatory manipulation of a novel chemically-defined neuron population in the hypothalamus results in a hypometabolic/hypothermic state over a very long period of time in mice. In this state, the metabolic rate decreases to one-third or less, but unlike an anesthetized state, the mice still react to changes in ambient temperature. Furthermore, the mice recover naturally from this state without any obvious abnormalities. This discovery provided important knowledge regarding the hibernation mechanism and knowledge for the development of methods for inducing an artificial hibernation-like state.

### SUMMARY OF INVENTION

The present invention provides a method for inducing a hibernation-like state and a device for the same.

The present inventors discovered that a hibernation-like state can be induced in subjects that are living, non-hibernating animals by applying, in the brain of the subjects, an excitatory stimulus to pyroglutamylated RFamide peptide (QRFP) gene-expressing neurons in regions of the hypothalamus including the anteroventral periventricular nucleus (AVPe), the medial preoptic area (MPA) and the periventricular nucleus (Pe).

According to the present invention, the following inventions are provided.

(1) A device for stimulating, in a brain of a living subject, pyroglutamylated RFamide peptide (QRFP)-producing neurons in at least one region selected from the group consisting of the anteroventral periventricular nucleus (AVPe), the medial preoptic area (MPA), and the periventricular nucleus (Pe), the device including:

a controller that transmits a control signal controlling the generation of a voltage;

a voltage generator that receives the control signal from the controller and generates a voltage;

a stimulation probe that is electrically connected proximally to the voltage generator and includes an electrical stimulation electrode at a distal end, the stimulation prove having sufficient length for accessing QRFP-producing neurons from a brain surface, and generating an electrical stimulus at the electrical stimulation electrode at the distal end through the voltage from the voltage generator;

an outside air temperature gauge;

a core body temperature gauge;

an exhaled gas analysis unit that measures oxygen concentration in exhaled gas; and

a recording unit that records a measured outside air temperature and at least one numeric value selected from the group consisting of a core body temperature and an oxygen concentration.

(2) A device for stimulating, in a brain of a living subject, pyroglutamylated RFamide peptide (QRFP)-producing neurons in at least one region selected from the group consisting of the anteroventral periventricular nucleus (AVPe), the medial preoptic area (MPA), and the periventricular nucleus (Pe), the device including:

a controller that transmits a control signal controlling a discharge of a QRFP-producing neuron stimulating compound;

a storage for the compound;

a compound transmitter that receives the control signal from the control unit and transmit the compound from the storage section of the compound;

a guide that includes a compound discharge port and a flow path for the compound to the discharge port, and delivers the compound to QRFP-producing neurons;

an outside air temperature gauge;

a core body temperature gauge;

an exhaled gas analysis unit that measures the oxygen concentration in exhaled gas; and

a recording unit that records a measured outside air temperature and at least one numeric value selected from the group consisting of a core body temperature and an oxygen concentration.

(3) The device according to (1) or (2), further including a determination unit that determines whether a subject is in a hypothermic state based on the outside air temperature and the core body temperature recorded in the recording unit.

(4) The device according to any one of (1) to (3), further including a determination unit that determines whether a subject is in a hypometabolic state based on the outside air temperature, the core body temperature, and the oxygen concentration recorded in the recording unit.

(5) The device according to any one of (1) to (4), further including a determination unit that determines whether a subject is in a hibernation-like state based on the outside air temperature, the core body temperature, and the oxygen concentration recorded in the recording unit.

(6) The device according to any one of (3) to (5), wherein the controller transmits a control signal for continuously or intermittently stimulating GRFP-producing neurons until the subject is determined to be in any one state selected from the group consisting of a hypothermic state, a hypometabolic state, and a hibernation-like state.

(7) A method of reducing a theoretical set-point temperature of a body temperature in a mammalian subject, the method including applying an excitatory stimulus to pyroglutamylated RFamide peptide (QRFP)-producing neurons.

(8) The method according to (7), wherein the QRFP-producing neurons are neurons in at least one region selected from the group consisting of the anteroventral periventricular nucleus (AVPe), the medial preoptic area (MPA) and the periventricular nucleus (Pe).

(9) The method according to (7) or (8), wherein the excitatory stimulus is a stimulus selected from the group consisting of chemical stimuli, magnetic stimuli and electrical stimuli.

(10) A method of screening a substance that applies an excitatory stimulus to pyroglutamylated RFamide peptide (QRFP)-producing neurons present in the anteroventral periventricular nucleus (AVPe), the medial preoptic area (MPA) and the periventricular nucleus (Pe) regions, the method including:

contacting a test compound and the QRFP-producing neurons with each other;

measuring an excitement of the QRFP-producing neurons; and

selecting a test compound that applies an excitatory stimulus to the QRFP-producing neurons.

(10a) A method of screening a substance that applies a specific excitatory stimulus to pyroglutamylated RFamide peptide (QRFP)-producing neurons present in the anteroventral periventricular nucleus (AVPe), the medial preoptic area (MPA) and the periventricular nucleus (Pe) regions, the method including:

expressing, in a cell, a receptor that is specifically expressed in a QRFP-producing neuron;

contacting a test compound and the cell with each other;

measuring an excitement of the QRFP-producing neurons; and

selecting a test compound that applies an excitatory stimulus to the QRFP-producing neurons.

(10b) A method of examining a substance that applies a specific excitatory stimulus to pyroglutamylated RFamide peptide (QRFP)-producing neurons present in the anteroventral periventricular nucleus (AVPe), the medial preoptic area (MPA) and the periventricular nucleus (Pe) regions, the method including:

providing pyroglutamylated RFamide peptide (QRFP)-producing neurons;

contacting a test compound and the cells with each other;

measuring an excitement of the QRFP-producing neurons; and

comparing the excitation of the QRFP-producing neurons before and after contact with the test compound to determine whether the test compound applies an excitatory stimulus to the QRFP-producing neurons.

(10c) A method of examining a substance that applies a specific excitatory stimulus to pyroglutamylated RFamide peptide (QRFP)-producing neurons present in the anteroventral periventricular nucleus (AVPe), the medial preoptic area (MPA) and the periventricular nucleus (Pe) regions, the method including:

administering a test compound in the anteroventral periventricular nucleus (AVPe), the medial preoptic area (MPA) and the periventricular nucleus (Pe) regions of a mammal;

measuring an excitement (electric potential, for example) of the QRFP-producing neurons; and

comparing the excitation of the QRFP-producing neurons before and after contact with the test compound to determine whether the test compound applies an excitatory stimulus to the QRFP-producing neurons.

(10d) A method of examining a test compound that induces hibernation, the method including:

administering a test compound in the anteroventral periventricular nucleus (AVPe), the medial preoptic area (MPA) and the periventricular nucleus (Pe) regions of a mammal (a non-human mammal, for example); and

confirming that the mammal begins hibernating.

(10e) The method according to (10d) above, wherein,

from a correlation between a core body temperature (for example, intestinal temperature) and an oxygen consumption amount of the mammal (for example, a non-human mammal), the core body temperature (theoretical set-point temperature) when the oxygen consumption amount is assumed to be 0 and ΔVO₂/ΔT_{B} (the feedback gain of heat production) are estimated, and

decrease in both the theoretical set-point temperature and the negative feedback gain of heat production, compared to those prior to administration of the test compound, indicates that the mammal is hibernating.

(10f) A method of testing whether a test compound induces hibernation in a mammal such as a human, the method further including:

providing an estimated value of a theoretical set-point temperature and an estimated value of a feedback gain of heat production of a human to which a test compound has been administered in the anteroventral periventricular nucleus (AVPe), the medial preoptic area (MPA) and the periventricular nucleus (Pe) regions, and an estimated value of the theoretical set-point temperature and an estimated value of the feedback gain of heat production of the human before administration of the test compound; and

confirming whether both the estimated value of the theoretical set-point temperature and the estimated value of the feedback gain of heat production after administration are reduced, compared to those prior to administration of the test compound; wherein

decrease in both the theoretical set-point temperature and the negative feedback gain of heat production, compared to those prior to administration of the test compound, indicates that the mammal is hibernating.

(10g) A method for determining (predicting, estimating, computationally and scientifically calculating) whether a test compound induces hibernation in a mammal such as human, the method including:

providing an estimated value of a theoretical set-point temperature and an estimated value of a feedback gain of heat production of a human to which a test compound has been administered in the anteroventral periventricular nucleus (AVPe), the medial preoptic area (MPA) and the periventricular nucleus (Pe) regions, and an estimated value of the theoretical set-point temperature and an estimated value of the feedback gain of heat production of the human before administration of the test compound; and

confirming whether both the estimated value of the theoretical set-point temperature and the estimated value of the feedback gain of heat production after administration are reduced, compared to the estimated value of the theoretical set-point temperature and the estimated value of the feedback gain of heat production prior to administration of the test compound; wherein

decrease in both the theoretical set-point temperature and the negative feedback gain of heat production, compared to those prior to administration of the test compound, indicates that the mammal is hibernating.

(10h) A method for determining (predicting, estimating, computationally and scientifically calculating) whether a test compound induces hibernation in a mammal such as human, the method including:

recording an oxygen consumption amount and a core body temperature recorded under at least two different ambient environment temperature conditions for both before administration and after administration of the test compound in a mammal such as a human to which a test compound has been administered in the anteroventral periventricular nucleus (AVPe), the medial preoptic area (MPA) and the periventricular nucleus (Pe) regions;

estimating a correlation between the oxygen consumption amount and the core body temperature for each of before administration and after administration of the test compound; and

determining, based on the estimated correlation, whether an extent of decrease in the oxygen consumption amount when the core body temperature has decreased is reduced after administration in comparison to before administration, and determining whether an estimated value of the core body temperature when the oxygen consumption amount is assumed to be 0 is reduced after administration in comparison to before administration; wherein

decrease in the extent of decrease in the oxygen consumption amount when the core body temperature has decreased is reduced in comparison to before administration, and in the estimated value of the core body temperature when the oxygen consumption amount is assumed to be 0 after administration of the test compound in comparison to before administration indicates that the mammal is hibernate.

(11) A method of determining (examining, predicting, estimating, computationally and scientifically calculating) whether a test compound induces hibernation in a mammal such as a human, the method including:

providing (or recording) an oxygen consumption amount and a core body temperature recorded under at least two different ambient environment temperature conditions for both before administration and after administration of the test compound in a mammal such as a human to which a test compound has been administered in the anteroventral periventricular nucleus (AVPe), the medial preoptic area (MPA) and the periventricular nucleus (Pe) regions;

estimating a correlation between the oxygen consumption amount and the core body temperature for each of before administration and after administration of the test compound; and

determining, based on the estimated correlation, whether an extent of decrease in the oxygen consumption amount when the core body temperature has decreased is reduced after administration in comparison to before administration, and determining whether an estimated value of the core body temperature when the oxygen consumption amount is assumed to be 0 is reduced after administration in comparison to before administration; wherein

decrease in the extent of decrease in the oxygen consumption amount when the core body temperature has decreased is reduced in comparison to before administration, and in the estimated value of the core body temperature when the oxygen consumption amount is assumed to be 0 after administration of the test compound in comparison to before administration indicates that the mammal is hibernate.

(12) A device for determining hibernation, the device including:

a recording unit which, with respect to a mammal such as a human to which a test compound has been administered in the anteroventral periventricular nucleus (AVPe), the medial preoptic area (MPA) and the periventricular nucleus (Pe) regions, records an oxygen consumption amount and a core body temperature recorded under at least two different ambient environment temperature conditions for both before administration and after administration of the test compound;

a calculation unit that estimates a correlation between the oxygen consumption amount and the core body temperature for each of before administration and after administration of the test compound, determines, based on the estimated correlation, whether an extent of decrease in the oxygen consumption amount when the core body temperature has decreased is reduced after administration in comparison to before administration, and determines whether an estimated value of the core body temperature when the oxygen consumption amount is assumed to be 0 is reduced after administration in comparison to before administration;

and a determination unit that determines that the mammal is hibernating if, after administration of the test compound, the extent of decrease in the oxygen consumption amount when the core body temperature has decreased is reduced in comparison to before administration, and the estimated value of the core body temperature when the oxygen consumption amount is assumed to be 0 is reduced after administration of the test compound in comparison to before administration.

### BRIEF DESCRIPTION OF DRAWINGS

FIG. 1a: FIGS. 1a to 1h pertain to activation of Qrfp-iCre neurons that reduce the hypothalamic body temperature and amount of energy consumption. FIG. 1a illustrates a strategy for chemically and genetically exciting iCre-positive neurons in a Qrfp-iCre mouse.

FIG. 1B: The chemical excitation of iCre-positive cells in Qrf-iCre mice was measured through infrared thermography, and as a result, was found to induce hypothermia. Hetero (Q-het) or homozygous (Q-homo) Qrfp-iCre mice having a heterozygous Rosa26^{dreaddm3} (M3) and/or Rosa26^{dreaeddm4} (M4) allele were subjected to the experiment.

FIG. 1c: Distribution of Q-neurons in the Qrfp-iCre mice. FIG. 1c is depicted by expression of GFP after injection of AAV10-DIO-GFP in the inner basal hypothalamus. Scale bar (horizontal image): 500 μm; insertion portion: 100 μm; coronal image: 200 μm. Pe: periventricular nucleus, AVPe: anteroventral Pe, MPA: medial preoptic area, LPO: lateral preoptic area, AHA: anterior hypothalmus, VMH: ventromedial hypothalamus, LHA: lateral hypothalamus, SON: supraoptic nucleus, DMH: dorsomedial hypothalamus, TMN: tuberomammillary nucleus, MM: medial mammillary nucleus, SCN: suprachiasmatic nucleus, VOLT: vascular organ of the lamina terminalis, TC: tuber cinereum, ARC: arcuate nucleus, third cerebroventricular optic nucleus.

FIG. 1d: Representative body temperature measurement results indicating the surface body temperature of Q-hM3D mice. CNO was injected into the peritoneum at 0 hours. Note that the temperature of the tail increases at 0.5 hours (indicated by the arrow).

FIG. 1e: Fos immunostaining of sliced specimens from the Q-hM3D mice 90 minutes after CNO IP. Scale bar: 100 μm.

FIG. 1f: Procedure for metabolic analysis by chemical and genetic activation of Q-neurons in Q-hM3D mice.

FIG. 1g: Progression over time of hypothermia and hypometabolism after activation of Cre-positive neurons through DREADD. Purple line, Q-hM3D mice; yellow line, Qrfp-iCre mice injected with AAV10-DIO-hM3Dq-mCherry into the lateral hypothalamus; black line, Qrfp-iCre mice injected with AAV-DIO-mCherry into the inner basal hypothalamus (negative control).

FIG. 1h: Q-neuron-induced hypometabolism (QIH) can last for several days and can be induced again by a CNO injection. The lines and shading of b and g indicate the average value and standard deviation of each group.

FIG. 2a: FIGS. 2a to 2l indicate the results of histological and functional analyses of Q-neuron projections. FIG. 2a illustrates a strategy for rendering an axonal projection pattern of Q-neurons rendered by expressing GFP in Q-neurons by injecting AAV-DIO-GFP into Qrfp-iCre mice.

FIG. 2b: Distribution of GFP-positive Q-neurons in the AVPe, MPA and Pe with respect to scale bar of 100 μm.

FIG. 2c: Distribution of axons generated from Q-neurons. Scale bar: 100 μm.

FIG. 2d: A cropped image of an image captured of the brain by the ScaleS method was clarified by the ScaleS method. Q-neurons in the AVPe and fibers in the DMH are shown.

FIG. 2e: An in situ hybridization analysis showing that a population of Q-neurons expresses Vgat and/or Vglut2 in Q-hM3D mice. Scale bar: 100 μm.

FIG. 2f: A highly magnified image of the rectangular area shown in FIG. 2e.

FIG. 2g: A monochrome image of the rectangular area in FIG. 2e.

FIG. 2h: A highly magnified image of the rectangular areas 1 to 3 illustrated in FIG. 2f, which illustrates Q-neurons expressing Vgat, Vglut2 or both. (1) Vgat⁺mCherry⁺; (2) Vglt2^{|}mCherry^{|}; (3) Vgat^{|}Vglt2^{|}mCherry^{|}.

FIG. 2i: Percentage of Vgat-positive neurons (quantity of 1291 in 1997 cells), Vglut2 (quantity of 359 in 1997 cells), and (quantity of 115 in 1197 cells) in mCherry expressing cells (counted in four sections prepared from two mice). Other mCherry-expressing cells do not express Vgat or Vglut2.

FIG. 2j: Strategy for light-generating excitation of Q-neurons or their axons in DMH and RPa, scale bar: 100 μm.

FIG. 2k: Transition in body temperature measured using a thermographic camera during optogenetic excitation in the DWI or RPa of Cre-positive cells or their axons in AVPe/MPA. Four shots of light stimulus are indicated by blue arrowheads. Each of the lines and shadings indicate the average value and standard deviation of each group. The lower panel shows representative thermographic images obtained by excitation of Q-neurons (AVPe/MPA). Note that the tail exhibits heat release after 5 minutes from the first light stimulus (arrow).

FIG. 2l: Estimated Tₛ 30 minutes after the fourth irradiation of the light stimulus. It should be noted that the effect of DMH fiber stimulation on Tₛ is almost comparable to the effect of excitation of the cell body in AVPe/MPA. Pe, periventricular nucleus; AVPe, anteroventricular Pe; VOLT, vascular organ of the lamina terminalis; MPA, medial preoptic area; VLPO, ventrolateral preoptic area; PVN, paraventricular hypothalamic nucleus; SON, supraoptic nucleus; DMH, dorsomedial hypothalamus; TMN, tuberomammillary nucleus; MM, medial mammillary nucleus; LC, locus coeruleus; PAG, periaqueductal grey; LPB, lateral parabrachial nucleus; RVLM, rostral ventrolateral medulla; third pallidal nucleus; pallidal nucleus; ventricle.

FIG. 3a: Hypometabolism induced by Q-neurons is accompanied by lowered set-point of body temperature. Transition in VO₂ and T_{B} in QIH at various TAs. QIH was induced in Q-hM3D mice by CNO injection. The lines and shading indicate the average value and standard deviation of each group.

FIG. 3b: Minimum T_{B} (left) and VO₂ (right) under normal and QIH conditions.

FIG. 3c: A schematic view of heat production and heat loss pathways in mammals. Heat loss is proportional to a difference between T_{A} and T_{B} at factor G. Heat production is governed by a difference between T_{R} and T_{A} at factor H.

FIG. 3d: Relationship between T_{B}-T_{A} and VO₂ at various TAs. The slope of the curve indicates G. The dots indicate recorded data, the thick lines are drawn from the median of the posterior G, and the thin lines are curves drawn from 500 Gs randomly selected from posterior samples.

FIG. 3e: Posterior distribution of estimated G (e) and difference in G from QIH to normal condition (f).

FIG. 3f: Posterior distribution of estimated G (e) and difference in G from QIH to normal condition (f).

FIG. 3g: Relationship between T_{B} and VO₂ at various TAs. The negative slope of the curve indicates H, and the x-intercept indicates TR. See FIG. 3d for a description of the dots and lines.

FIG. 3h: Distribution of estimated H (h) and difference in H from QIH to normal condition (i).

FIG. 3i: Distribution of estimated H (h) and difference in H from QIH to normal condition (i).

FIG. 3j: Distribution of estimated TR (j) and difference in TR from QIH to normal condition (k).

FIG. 3k: Distribution of estimated TR (j) and difference in TR from QIH to normal condition (k).

FIG. 3l: Metabolic transition of QIH within an individual. Upper row shows transition of animal posture at various TAs during QIH. Second row is timewise magnification of third row, both showing metabolic transitions in one representative animal. Note that the mouse shows a curled-up posture during FIT at T_{A}=28° C. (B), while it shows an extended posture during QIH at T_{A}=28° C. (D). Even during QIH when T_{A} is lowered to 12° C., the animal assumes a curled-up posture, as in FIT (E), indicating that the animal was trying to avoid heat loss.

FIG. 4a: FIGS. 4a to 4g demonstrate that Q neurons play a role in inducing fasting-induced torpor in mice. FIG. 4a illustrates a strategy for suppressing Q neuronal function. Left panel, experimental procedure. Right panels, expression of TeTxLC-eYFP in the AVPe/MPA shown by immunostaining with anti-GFP antibody. Scale bar, 100 μm.

FIG. 4b: Schematic view of the FIT experiment.

FIG. 4c: FIT was disrupted by expressing TeTxLC in Q neurons. Note that a rapid oscillatory decrease in metabolism was never seen in these mice.

FIG. 4d: Minimum VO₂ during 24-36 h and 36-48 h periods was compared between control and TeTxLC mice. Suppression of Q neurons blocked the VO₂ decrease normally seen in FIT. The estimated difference in minimum VO₂ between control and TeTxLC mice was [0.01, 0.80] ml/g/h at 24-36 h and [0.36, 1.16] ml/g/h at 36-48 h. The smaller SD of T_{B} and VO₂ in TeTxLC mice indicates that Q neurons are involved in the abrupt change in metabolism including the oscillatory changes during FIT. The “>” and “<” signs denote whether the 89% HPDI of the difference in estimated minimum value or the standard deviation from TeTxLC to control mice is negative or positive, respectively.

FIG. 4e: Indicates that FIT was induced in control, Qrfp-iCre hetero and homo mice, showing that lack of QRFP peptide did not affect FIT.

FIG. 4f: Procedure for depicting input neurons that make mono-synaptic contact with Q neurons using rabies virus vector.

FIG. 4g: Distribution of input neurons of Q neurons. Arrows show starter cells. Scale bars, 100 μm.

FIG. 4h: Brain regions containing input neurons. Scale bars, 100 μm. Pe, periventricular nucleus; AVPe, anteroventricular Pe; MPA, medial preoptic area; VOLT, vascular organ of the lamina terminalis; MnPO, median preoptic area; VMPO, ventromedial preoptic area; VLPO, ventrolateral preoptic area; PVN, paraventricular hypothalamic nucleus; TC, tuber cinereum; opt, optic tract; ac, anterior commissure; f, fornix; 3V, third ventricle.

FIG. 5: FIG. 5 illustrates an overview of the device according to a first embodiment.

FIG. 6: FIG. 6 illustrates an overview of the device according to the first embodiment.

FIG. 7: FIG. 7 illustrates an overview of the device according to a second embodiment.

FIG. 8 illustrates an overview of additional configurations of the devices of the first and second embodiments.

### DETAILED DESCRIPTION OF THE INVENTION

Herein, “subject” refers to a human and non-human mammals, including, for example, rats, and non-human primates such as monkeys, gorillas, chimpanzees, orangutans and bonobos.

Herein, “hypothalamus” is an organ that is present in the diencephalon, and is the center of regulation of endocrine and autonomic functions. Herein, “Q-neurons” are neurons that are present in an inner region of the hypothalamus, namely, the regions of the anteroventral periventricular nucleus (AVPe), the medial preoptic area (MPA), and the periventricular nucleus (Pe), and these Q-neurons produce pyroglutamylated RFamide peptide (QRFP). The pyroglutamylated RFamide peptide (QRFP) is a neuropeptide identified as an endogenous ligand of the GPR103 receptor. QRFPs are strongly expressed in the hypothalamus and are thought to be involved in the regulation of sleep and wakefulness, as such neurons have been shown to clearly have an effect of enhancing the arousal system.

Herein, “T_{A}” means the ambient environment temperature (° C.) of the subject, “T_{B}” means the core body temperature (° C.), and “T_{R}” means the theoretical set-point temperature (° C.). “VO₂” means the amount of oxygen consumed by the subject. T_{R} is the body temperature determined as T_{B} when the correlation between VO₂ and T_{B} when T_{A} is varied is determined and VO₂ is zero. T_{B} is the temperature inside the body, and is not at the temperature of the body surface, which is affected by the outside air temperature. For example, T_{B} in a human can be defined as the temperature in the rectum, the temperature in the esophagus, the temperature of bladder, or the temperature of blood in the pulmonary artery. The negative feedback gain (H) of heat production indicates exothermic efficiency, and is determined by H=ΔVO₂/ΔT_{B}.

Herein, “hibernation” is a hypothermic and hypometabolic state observed in mammals. “Daily torpor” is a short-term hypometabolic state. Hibernation and daily torpor differ in that in daily torpor, H is reduced with almost no reduction in T_{R}, whereas in hibernation, both T_{R} and H are significantly reduced. Herein, a “hibernation-like state” means a state in which T_{R} and H are both significantly reduced in association with a reduction in T_{A}. Herein, a “non-hibernating animal” refers to an animal that does not have a mode of hibernation during the winter or when fasting.

Herein, “exhalation” is a breath exhaled by a subject. Herein, the oxygen concentration is an index indicating the amount of oxygen per volume. The unit of oxygen concentration can be, for example, % or mmHg. Herein, the “oxygen consumption amount” (VO₂) is the amount of oxygen consumed per unit time, and is calculated from the oxygen concentration contained in exhalation and inhalation. The oxygen consumption amount fluctuates due to body weight, and thus may be calculated with corrections in the per unit volume (e.g., per kg and per g).

The present inventors discovered that a hibernation-like state can be induced in subjects that are living, non-hibernating animals by applying, in the brain of the subjects, an excitatory stimulus to pyroglutamylated RFamide peptide (QRFP)-producing neurons in at least one region of the hypothalamus selected from the anteroventral periventricular nucleus (AVPe), the medial preoptic area (MPA) and the periventricular nucleus (Pe).

Thus, according to the present invention, provided is a method for inducing a hibernation-like state in a subject that is a living, non-hibernating animal, the method including applying an excitatory stimulus to pyroglutamylated RFamide peptide (QRFP)-producing neurons in at least one region of the hypothalamus, selected from the group consisting of the anteroventral periventricular nucleus (AVPe), the medial preoptic area (MPA) and the periventricular nucleus (Pe).

Excitatory stimuli can be triggered by stimulation using a deep brain electrode or by stimulation using an activator of QRFP-producing neurons.

According to the present invention, provided is a device (hereinafter, may be referred to as the “device of the present invention”) for stimulating pyroglutamylated RFamide peptide (QRFP)-producing neurons in at least one region selected from the group consisting of the anteroventral periventricular nucleus (AVPe), the medial preoptic area (MPA) and the periventricular nucleus (Pe) in the brain of a living subject.

(A1) A device of the present invention may include:

a controller that transmits a control signal controlling the generation of a voltage;

a voltage generator that receives a control signal from the controller and generates a voltage; and

a stimulation probe that is electrically connected proximally to the voltage generator and including an electrical stimulation electrode at a distal end, the stimulation prove having sufficient length for accessing QRFP-producing neurons from a brain surface, and generating an electrical stimulus at the electrical stimulation electrode at the distal end through voltage from the voltage generator.

Through such configuration, the device of the present invention can electrically apply an excitatory stimulus to QRFP-producing neurons. Alternatively, from the perspective of applying a chemical stimulus instead of an electrical stimulus,

(A2) the device of the present invention may include:

a controller that transmits a control signal controlling a discharge of a QRFP-producing neuron stimulating compound;

a storage for the compound; and

a compound discharge unit that receives a control signal from the controller and discharges the compound from the storage of the compound.

(B) The device of the present invention may further include:

an outside air temperature gauge;

a core body temperature gauge;

an exhaled gas analysis unit that measures an oxygen concentration in exhaled gas; and a recording unit that records a measured outside air temperature and at least one numeric value selected from the group consisting of a core body temperature and an oxygen concentration. In the configuration (B) of the device of the present invention, whether the core body temperature (T_{B}) of the subject decreases in association with a decrease in the outside air temperature (T_{A}) can be examined, the oxygen consumption amount of the subject can be determined from the exhaled gas analysis results, and the theoretical set-point temperature (T_{R}) and negative feedback gain (H) of heat production can be determined. Such an configuration allows the device of the present invention to determine whether a hibernation state has been induced in a subject.

The device of the present invention will be described in detail below.

### First Embodiment

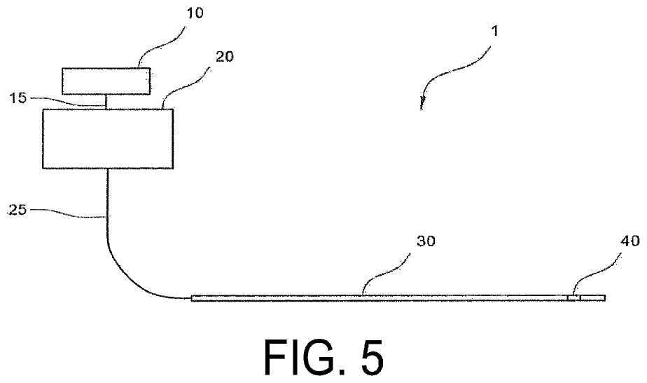

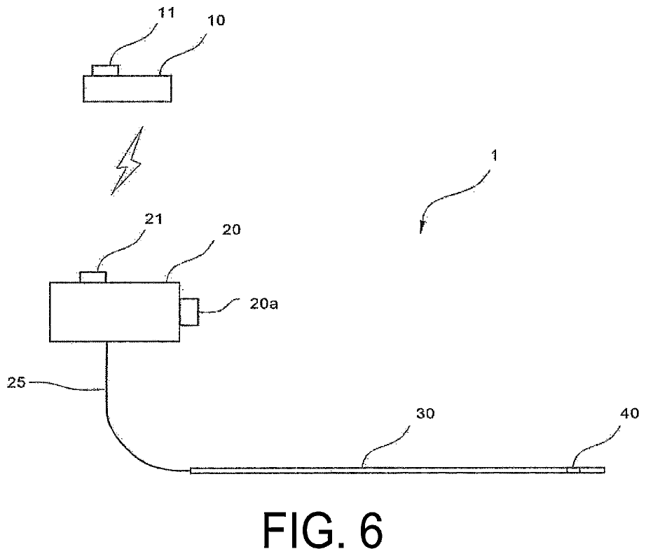

In a first embodiment, the device of the present invention has the configuration of (A1) above. Through this configuration, the device of the present invention induces a hibernation state in a living subject by electrically stimulating the QRFP-producing neurons of the brain of the subject. The first embodiment will be described below with reference to FIGS. 5 and 6.

(A1) A device 1 of the present invention includes:

a controller 10 that transmits a control signal controlling the generation of a voltage;

a voltage generator 20 that receives a control signal from the controller and generates a voltage; and

a stimulation probe 30 that is electrically connected proximally to the voltage generator, and includes an electrical stimulation electrode at a distal end, the stimulation prove having sufficient length for accessing QRFP-producing neurons from a brain surface, and generating an electrical stimulus at the electrical stimulation electrode 40 at the distal end through voltage from the voltage generator.

In the device 1 of the present invention, the controller 10 transmits a control signal that controls voltage generation. The controller 10 may include a control element (microprocessor and power source or battery) The control signal can control voltage generation for once or multiple times through a single control signal. Alternatively, the control signal can be transmitted multiple times to control voltage generation for multiple times. The control signal can apply a voltage stimulus in one step, but for example, the control signal may control voltage generation such that a stimulus is applied a plurality of times until a hibernation-like state is induced in the subject (of course, after the hibernation-like state has been induced, the stimulus may or may not be applied).

In the device 1 of the present invention, the voltage generator 20 is electrically coupled to the controller 10 through wiring 15, and can receive a control signal from the controller 10 and generate a voltage. The voltage may be, for example, a voltage of from 0 to 5 volts (V), and may be varied, for example, in units of 0.1 volts. The voltage can be, for example, a pulsed voltage. The pulse width can be set to, for example, several tens of microseconds, and the stimulus frequency can be, for example, tens to hundreds of pps. The voltage may, for example, begin from 1 volt, and then be adjusted so as to increase until the effect is observed.

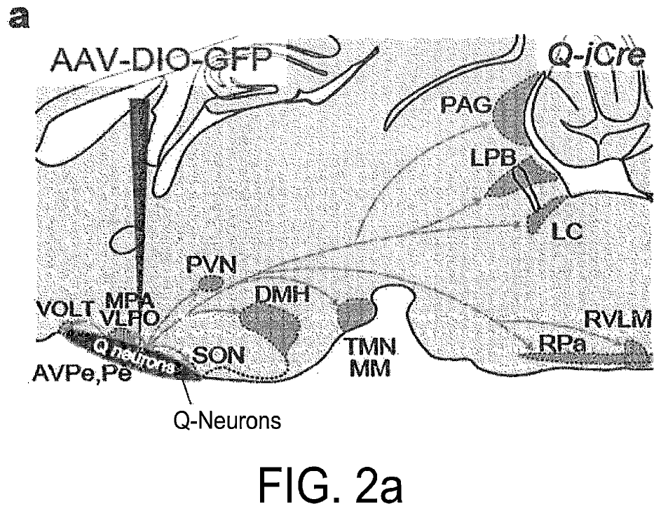

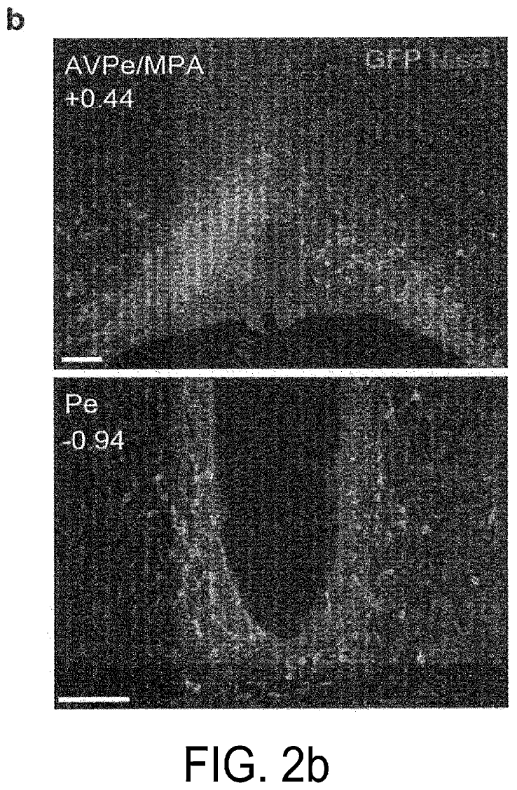

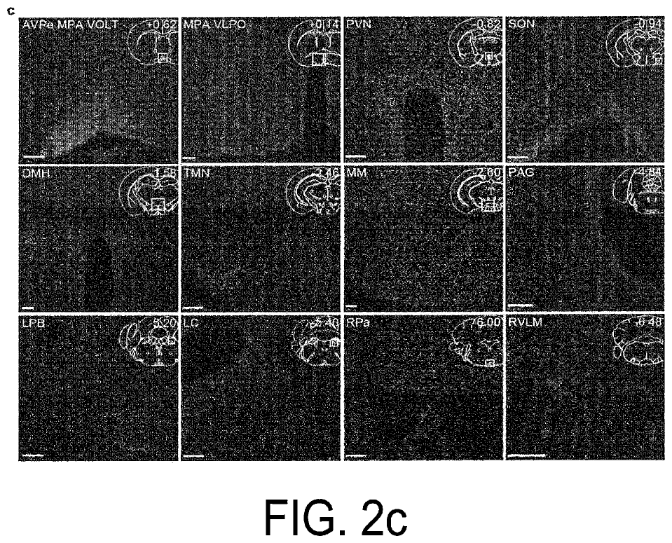

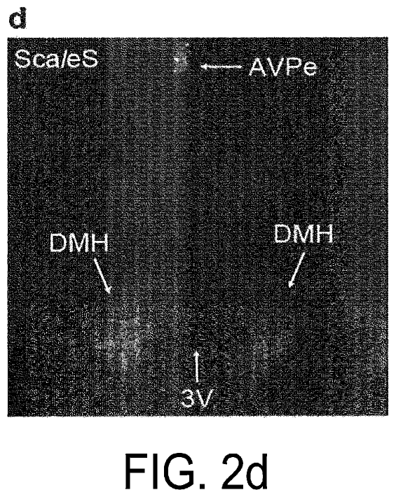

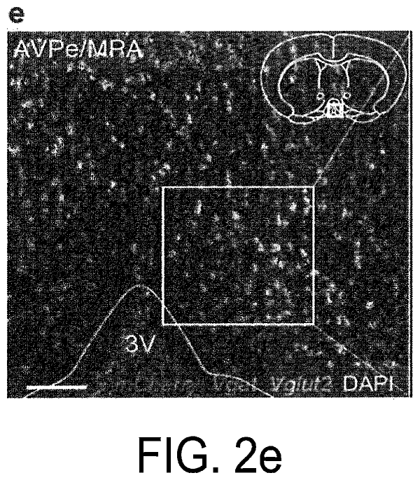

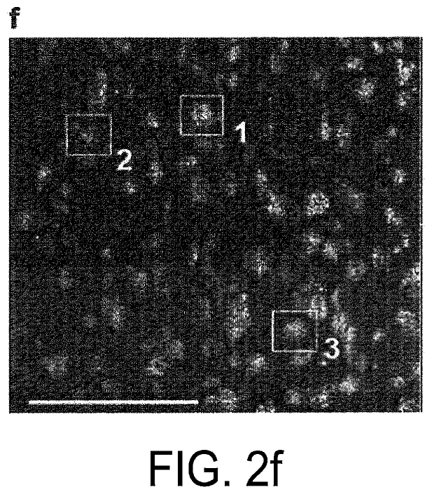

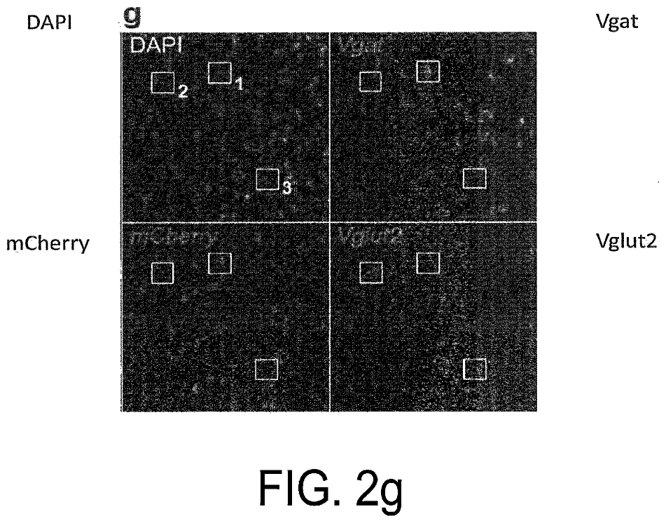

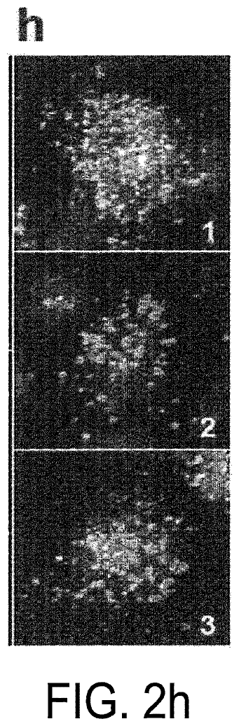

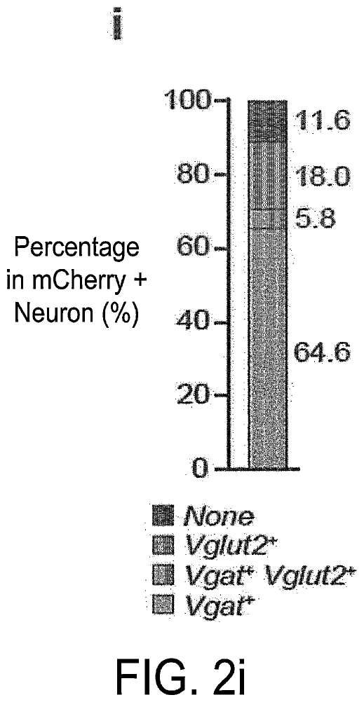

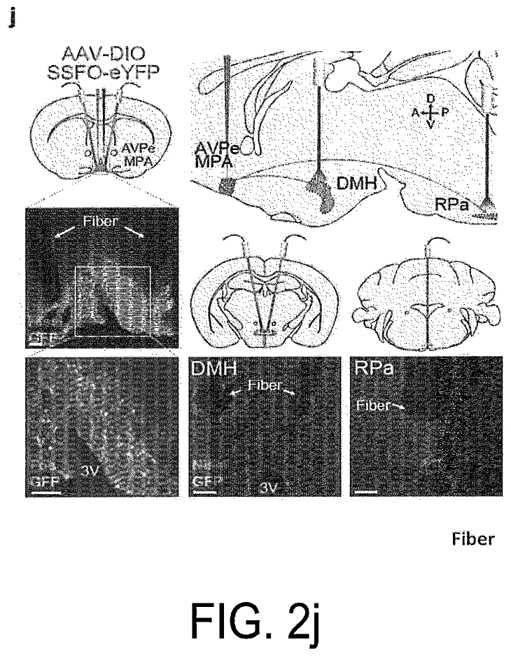

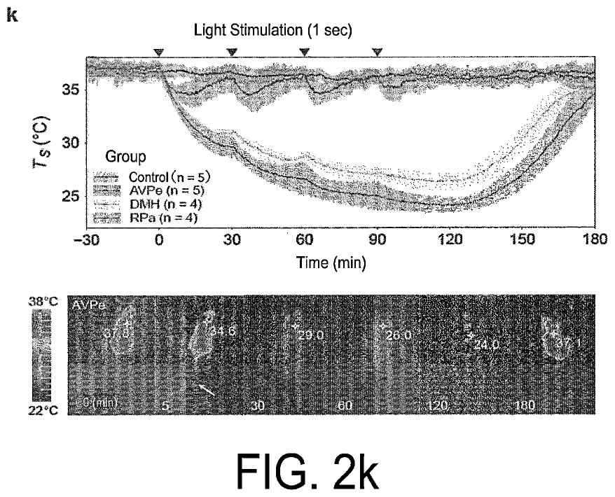

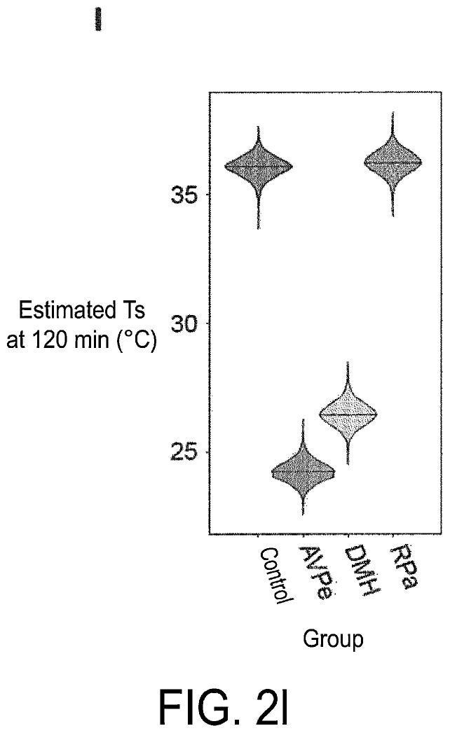

An example was described in which the controller 10 and the voltage generator 20 are connected through the wiring 15. However, with the device 1 of the present invention, instead of connection through the wiring 15, as illustrated in FIG. 2, the controller 10 and the voltage generator 20 may be capable of wirelessly communicating between a control signal transmission unit 11 included in the controller 10 and a control signal reception unit 21 included in the voltage generator. In this aspect, the voltage generator 20 can have a battery 20a. The battery 20a may be rechargeable in a non-contact manner. When charging is possible in a non-contact manner, the battery 20a can be charged from outside the body even when present in the body.

The voltage generator 20 transmits, through an extension cable 25, a voltage generated by the voltage generator 20 to a stimulation probe 30 and a stimulation electrode 40 present at the tip end. The distal end (that is, the tip end) of the stimulation probe 30 has the stimulation electrode 40, and the stimulation electrode 40 can apply a voltage to the tissue of the brain.

The stimulation probe 30 may be inserted into the brain through a stereotaxic surgery such that the stimulation electrode 40 precisely reaches the QRFP-producing neurons. In stereotaxic surgery, the head is fixed with a measuring frame, and the electrode is inserted with a precision of 1 mm or less to a position at which the electrode is to be inserted, the position being determined through a CT scan or MRI. From the perspective of the stereotaxic surgery, the stimulation probe 30 is formed from a material (e.g., a hard material such as tungsten) that is hard enough such that the probe does not bend or stretch when piercing towards a deep brain section. The stimulation probe 30 is not particularly limited, and for example, may have a diameter from approximately 1 μm to 1 mm, or from 1 mm to 2.5 mm. The stimulation probe 30 has one or more (e.g., two, three or four) stimulation electrode(s) 40 at a distal end. The stimulation electrode 40 may have a length of approximately 1 to 5 mm in the long axis direction of the stimulation probe 30. When the stimulation probe 30 has a plurality of stimulation electrodes 40, the stimulation electrodes 40 may be disposed at an interval of approximately 1 mm to 1.5 mm, for example, although this interval is not particularly limited. Each of the stimulation electrodes 40 may be controlled collectively by one control signal, or each may preferably be separately controlled by individual control signals. When each stimulation electrode 40 is controlled separately by an individual control signal, the brain can be stimulated with a voltage that is selectively generated at an optimal electrode in relation to the insertion position of the electrode.

The device 1 of the present invention induces a hibernation-like state in the subject, and does not need to be portable. Here, portable means moving together with the movement of a subject in relation to a footing (e.g., the ground or a floor of a vehicle when riding in the vehicle) where the subject is located. Accordingly, the device of the present invention may be fixed to an installation site. The device of the present invention can be connected to a power source, and therefore, for example, the device need not include a battery or rechargeable battery.

### Second Embodiment

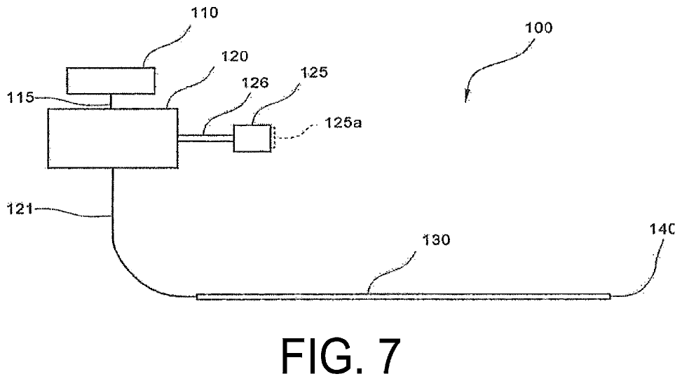

In the first embodiment, a device that electrically stimulates a deep section of the brain was disclosed, but the second embodiment pertains to a device that chemically stimulates a deep brain section. The second embodiment will be described below with reference to FIG. 7.

In the second embodiment, a device 100 of the present invention includes:

a controller 110 that transmits a control signal controlling the discharge of a QRFP-producing neuron stimulating compound;

a storage 125 for the compound;

a compound transmitter that receives the control signal from the control unit and transmits the compound from the storage section 125 of the compound; and

a guide 130 including a compound discharge port 140 and a flow path for the compound to the discharge port 140, and delivers the compound to the QRFP-producing neurons. In the device 100 of the present invention, the controller 110 is electrically connected to the compound transmitter 120 through wiring 115. The compound transmitter 120 receives a control signal from the controller 110, and according to the control signal thereof, discharges the compound accumulated in the storage 125 into the brain from the compound discharge port 140 from the storage 125 through a flow path 126 and a flow path 121 and the guide 130. The compound may be in the form of a solution in which the compound is dissolved in a solvent, and may be fed to the compound discharge port 140 by a transmit mechanism using the compound transmitter 120. The storage 125 of the compound may have a compound inlet 125a through which a compound is introduced from the outside. The compound inlet 125a can supply the compound into a compound storage. The compound storage 125 may be exposed outside of the body. However, if the compound storage 125 is exposed outside of the body, the compound storage 125 is maintained under sterile conditions. The controller 110 transmits, to the compound transmitter 120, a control signal that causes, for example, transmiting of 1 μL to 100 μL of the compound per a single transmission of the compound.

The guide 130 can be inserted into the brain through a stereotaxic surgery such that the compound discharge port 140 precisely reaches the QRFP-producing neurons. In stereotaxic surgery, the head is fixed with a measuring frame, and the electrode is inserted with a precision of 1 mm or less to a position at which the electrode is to be inserted, the position being determined through a CT scan or MRI. From the perspective of the stereotaxic surgery, the guide 130 is formed from a material (e.g., a hard material such as tungsten) that is hard enough such that the guide 130 does not bend or stretch when piercing towards a deep brain section. The stimulation probe 30 can have a diameter from approximately 1 mm to 2.5 mm, for example.

The device 100 of the present invention induces a hibernation-like state in a subject, and need not be portable. Here, portable means that moving together with the movement of a subject in relation to a footing (e.g., the ground or a floor of a vehicle when riding in the vehicle) where the subject is located. Accordingly, the device of the present invention may be fixed to an installation location (e.g., a bed on which a subject lies down, or a floor on which the bed is disposed). The device of the present invention can be connected to a power source, and therefore, for example, the device need not include a battery or rechargeable battery.

### Additional Configurations

The device 1 of the first embodiment and the device 100 of the second embodiment may further include the configuration of (B):

an outside air temperature gauge 50;

a body temperature gauge 60;

an exhaled gas analysis unit 70 that measures an oxygen concentration in exhaled gas; and

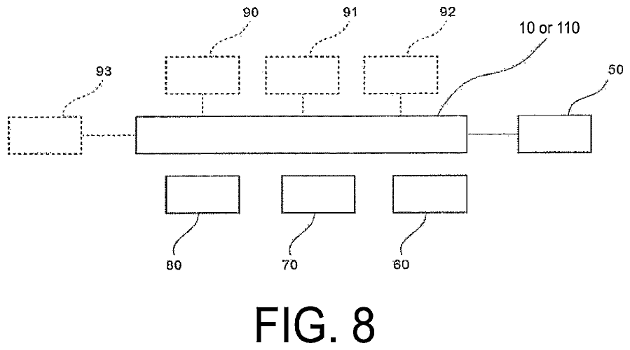

a recording unit 80 that records a measured outside air temperature and at least one numeric value selected from the group consisting of a body temperature and an oxygen concentration (here, the body temperature gauge is preferably a core body temperature gauge that measures the core body temperature of the subject). As illustrated in FIG. 8, the above-described (B) may include, for example, the controller 10 or the controller 110 (here, while illustrating has been omitted in FIG. 8, the controllers 10 and 110 are each connected, by wire or wirelessly, to the voltage generator 20 as described in the first embodiment and the second embodiment, respectively). When inducing a hibernation-like state in a subject, the outside air temperature (or ambient temperature of the subject) (T_{A}) is lowered, and the core body temperature (T_{B}) and metabolism are reduced. Accordingly, by including an outside air temperature gauge that measures the outside air temperature (or ambient temperature of the subject) and a body temperature gauge (preferably a core body temperature gauge), the device of the present invention can monitor the relationship between the body temperature of the subject (preferably, the core body temperature) and the outside air temperature.

In addition, by including an exhaled gas analysis unit 70 that measures the oxygen concentration in an exhaled gas, the device of the present invention can estimate the amount of oxygen consumption (VO₂) by the subject, and can estimate the metabolic state of the subject from the oxygen consumption amount (VO₂).

The present invention can also estimate, based on the core body temperature (T_{B}) and the oxygen consumption amount (VO₂), the theoretical set-point temperature (T_{R}) of the body temperature and the feedback gain (II) of heat production. The theoretical set-point temperature (T_{R}) is determined as an estimated value of the core body temperature (T_{B}) when the oxygen consumption amount (VO₂) is set to 0 by determining the relationship between the core body temperature (T_{B}) and the oxygen consumption amount (VO₂) while varying the outside air temperature (or ambient temperature of the subject) (T_{A}). The relationship between the core body temperature (T_{B}) and the oxygen consumption amount (VO₂) can be determined, for example, through linear regression. Also, the feedback gain (H) of heat production can be determined from H=ΔVO₂/ΔT_{B}.

The device of the present invention may further include a recording unit 80 that records a measured outside air temperature and at least one numeric value selected from the group consisting of the body temperature (preferably, the core body temperature) and the oxygen concentration. The device of the present invention may further include an oxygen consumption amount determination unit 90 that determines the amount of oxygen consumption of the subject from the oxygen concentration in the exhaled gas. The device of the present invention may further include an estimating unit 91 that estimates the theoretical set-point temperature (T_{R}) of the body temperature and the feedback gain (II) of heat production. The device of the present invention may further include a determination unit 92 that determines, based on the theoretical set-point temperature (T_{R}) of the body temperature and the feedback gain (H) of heat production, whether a hibernation-like state has been induced in the subject. The device of the present invention may further include an output unit 93 that outputs information regarding whether a hibernation-like state has been induced. Examples of the output unit 93 include a display that displays the information and/or a printer that prints the information. Examples of information regarding whether a hibernation-like state has been induced include information indicating that a hibernation-like state was induced, and information indicating that a hibernation-like state was not induced, and the information thereof may be output by the output unit 93.

### Third Embodiment

According to the present invention,

provided is a device for determining hibernation, the device including:

a recording unit which records an oxygen consumption amount (VO₂) and a core body temperature (T_{B}) recorded under at least two different ambient environment temperature (T_{A}) conditions for both before administration and after administration of the test compound in a mammal such as a human to which a test compound has been administered in regions of the anteroventral periventricular nucleus (AVPe), the medial preoptic area (MPA), and the periventricular nucleus (Pe); and

a calculation unit that estimates a correlation between the oxygen consumption amount and the core body temperature for each of before administration and after administration of the test compound, determines, based on the estimated correlation, whether an extent of decrease in the oxygen consumption amount when the core body temperature has decreased is reduced after administration in comparison to before administration, and determines whether an estimated value of the core body temperature when the oxygen consumption amount is assumed to be 0 is reduced after administration in comparison to before administration;

and further including a determination unit that determines that the mammal is hibernating if, after administration of the test compound, the extent of decrease in the oxygen consumption amount when the core body temperature has decreased is reduced in comparison to before administration, and, after administration of the test compound, the estimated value of the core body temperature when the oxygen consumption amount is assumed to be 0 is reduced in comparison to before administration.

The recording unit records the oxygen consumption amount (VO₂) and the core body temperature (T_{B}) recorded under at least two different ambient environment temperature (T_{A}) conditions. The recording unit stores one VO₂ and one T_{B} in association with one T_{A}. The recorded oxygen consumption amount (VO₂) and core body temperature (T_{B}) are read from the recording unit and then transmitted to the calculation unit, and the correlation between the oxygen consumption amount and the core body temperature is estimated by the calculation unit. In certain aspects, the correlation is linear. After the correlation has been estimated, the calculation unit determines whether the extent of decrease in oxygen consumption when the core body temperature has been reduced is reduced after administration in comparison to before administration, and determines whether the estimated value of the core body temperature when the oxygen consumption amount is assumed to be 0 is reduced after administration in comparison to before administration. On the basis of the determination by the calculation unit, the determination unit determines that the mammal is hibernating if, after administration of the test compound, the extent of decrease in the oxygen consumption amount when the core body temperature has decreased is reduced in comparison to before administration, and, after administration of the test compound, the estimated value of the core body temperature when the oxygen consumption amount is assumed to be 0 is reduced in comparison to before administration. The determination unit can be configured to not determine that the mammal is hibernating (or to determine that the mammal is not hibernating) for a case in which after administration of the test compound, the extent of decrease in the oxygen consumption amount when the core body temperature has decreased is not reduced in comparison to before administration, or, after administration of the test compound, the estimated value of the core body temperature when the oxygen consumption amount is assumed to be 0 is not reduced in comparison to before administration.

The device for determining hibernation according to the third embodiment of the present invention may further include a core body temperature gauge and an exhaled gas analysis unit that measures the oxygen concentration in exhaled gas. The device according to the third embodiment may further include an output unit that receives, from the determination unit, the determination information pertaining to hibernation and outputs the information. The information output unit may be a user interface, such as a display, may be a recording device that records to a non-volatile memory such as USB memory or an SD card, may be an information transmission device for wireless communication to the outside, or may be a printing device such as a printer for printing onto a medium such paper.

The device of the first embodiment or the second embodiment may further include the device for determining hibernation according to the third embodiment.

### Stimulation Method of the Present Invention

According to the present invention, provided is a method of reducing, in a subject, a theoretical set-point temperature of body temperature and/or a feedback gain of heat production. According to the present invention, a method of inducing a hibernation-like state in a subject is provided.

The method according to the present invention includes applying an excitatory stimulus to pyroglutamylated RFamide peptide (QRFP)-producing neurons. According to the present invention, also provided is a method for reducing, in a subject, a feedback gain of heat production, the method including applying an excitatory stimulus to pyroglutamylated RFamide peptide (QRFP)-producing neurons. According to the present invention, also provided is a method for reducing, in a subject, a theoretical set-point temperature of a body temperature and a feedback gain of heat production, the method including applying an excitatory stimulus to pyroglutamylated RFamide peptide (QRFP)-producing neurons. According to the present invention, also provided is a method for inducing a hibernation-like state in a subject, the method including applying an excitatory stimulus to pyroglutamylated RFamide peptide (QRFP)-producing neurons.

In the method of the present invention, pyroglutamylated RFamide peptide (QRFP)-producing neurons can be stimulated using, for example, a device of the present invention. In the method of the present invention, a voltage can be loaded onto QRFP-producing neurons, thereby stimulating the QRFP-producing neurons. In the method of the present invention, a stimulus can be applied to the QRFP-producing neurons by expressing, specifically in the QRFP-producing neurons, a receptor (e.g., hM3Dq) using the DREADD method, and administering a ligand (e.g., clozapine-N-oxide (CNO)) for the receptor thereof. The hM3Dq can be expressed in the QRFP-producing neurons by infecting the QRFP-producing neurons of the subject with a virus (e.g., adenovirus, adeno-associated virus, etc.) having a gene encoding hM3Dq, which is operably linked to a QRFP promoter. The CNO can be administered to the brain, for example, by the device of the invention.

In the method of the present invention, pyroglutamylated RFamide peptide (QRFP)-producing neurons can also be stimulated using an activator of the neurons. The activator can be screened using QRFP neurons or can be searched for using cultured cells in which receptors that express on QRFP neurons have been forcibly expressed. A neuron activator may be administered locally to QRFP-producing neurons using an applicator. The QRFP-producing neuron-specific activator may be administered through intracerebroventricular administration, intrathecal administration, or systemic administration such as intravenous administration.

The method of the present invention may further include lowering the outside air temperature. Through this, the T_{B} of the subject can be reduced. It is thought that when the T_{B} is reduced in a hibernation-like state, a hypometabolic state is created, energy consumption is reduced, and life can be maintained.

The method of the present invention may further include measuring the core body temperature (T_{B}) of the subject. The method of the present invention may further include measuring the oxygen concentration in exhaled air of the subject.

The method of the present invention may further include estimating the oxygen consumption amount (VO₂) of the subject. The oxygen consumption amount (VO₂) of the subject can be estimated, for example, based on the difference in oxygen concentrations between inhaled air and exhaled air.

The method of the present invention includes: with respect to a mammal such as a human to which a test compound has been administered in regions of the anteroventral periventricular nucleus (AVPe), the medial preoptic area (MPA), and the periventricular nucleus (Pe), providing (or recording) an oxygen consumption amount and a core body temperature recorded under at least two different ambient environment temperature conditions for both before administration and after administration of the test compound;

estimating a correlation between the oxygen consumption amount and the core body temperature for each of before administration and after administration of the test compound; and

determining, based on the estimated correlation, whether an extent of decrease in the oxygen consumption amount when the core body temperature has decreased is reduced after administration in comparison to before administration, and determining whether an estimated value of the core body temperature when the oxygen consumption amount is assumed to be 0 is reduced after administration in comparison to before administration; and

if, after administration of the test compound, the extent of decrease in the oxygen consumption amount when the core body temperature has decreased is reduced in comparison to before administration, and the estimated value of the core body temperature when the oxygen consumption amount is assumed to be 0 is reduced after administration of the test compound in comparison to before administration, the condition thereof indicates that the mammal is hibernating.

In this aspect, the method of the present invention may further include estimating the theoretical set-point temperature (T_{R}) of the body temperature of the subject. The theoretical set-point temperature (T_{R}) is determined as an estimated value of the core body temperature (T_{B}) when the oxygen consumption amount (VO₂) is set to 0 by determining the relationship between the core body temperature (T_{B}) and the oxygen consumption amount (VO₂) while varying the outside air temperature (or ambient temperature of the subject) (T_{A}). The relationship between the core body temperature (T_{B}) and the oxygen consumption amount (VO₂) can be determined, for example, through linear regression.

The method of the present invention may further include estimating the feedback gain (H) of heat production of the subject. The feedback gain (H) of heat production can be determined from H=ΔVO₂/ΔT_{B}.

The method of the present invention may further include determining whether the subject is in a hibernation-like state. Whether the subject is in hibernation-like state can be determined on the basis of whether the theoretical set-point temperature (T_{R}) of the body temperature and the feedback gain (H) of heat production are both reduced when the outside air temperature is reduced. If the theoretical set-point temperature (T_{R}) of the body temperature and the feedback gain (H) of heat production are both reduced when the outside air temperature is reduced, it can be determined that the subject is in a hibernation-like state.

The hibernation-like state may be beneficial in improving life protection functions by decreasing the metabolism of a living body.

### Screening System of the Present Invention

According to the present invention, a method of screening a substance that applies an excitatory stimulus to pyroglutamylated RFamide peptide (QRFP)-producing neurons present in regions of the anteroventral periventricular nucleus (AVPe), the medial preoptic area (MPA) and the periventricular nucleus (Pe) is provided, the method including:

bringing a test compound and the QRFP-producing neurons that have been isolated into contact with each other;

measuring an excitement of the QRFP-producing neurons; and

selecting a test compound that applies an excitatory stimulus to the QRFP-producing neurons. The method can be an in vitro method.

The excitement of the QRFP-producing neurons can be measured electrically. The excitation of the neurons can be measured by, for example, measuring, as an index, the depolarization of a membrane potential through an electrophysiological technique using a conventional method. The membrane potential can be measured by, for example, a neural recording method such as a microelectrode method, or by a patch clamp method, or may be measured using a fluorescent probe for measuring the membrane potential. The fluorescent probe for measuring the membrane potential is not particularly limited, and examples include 4-(4-(didecylamino) styryl)-N-methylpyridinium iodide(4-Di-10-ASP), bis-(1,3-dibutylbarbituric acid)trimethine oxonol (DiSBAC 2 (3)), 3,3′-dipropylthiadicarbocyanine iodide (DiSC 3 (5)), 5,5′,6,6′-tetrachloro-1,1′,3,3′-tetraethyl benzimidazolyl carbocyanine iodide (JC-1) and rhodamine 123. Excitation of the neurons can also be measured chemically. When neurons are excited, the intracellular calcium concentration increases. For example, excitation of the neurons can be measured using a calcium concentration indicator. As the calcium concentration indicator, a variety of probes such as 1-[6-amino-2-(5-carboxy-2-oxazolyl)-5-benzofuranyloxy]-2-(2-amino-5-methylphenoxy)ethane-N,N,N′,N′-tetraacetic acid, and pentaacetoxy methyl ester (Fura 2-AM) are known and can be used in the present invention.

QRFP-producing neurons are neurons that are present in the regions of the anteroventral periventricular nucleus (AVPe), the medial preoptic area (MPA), and the periventricular nucleus (Pe), and neurons of an established cell line can be used. As neurons of an established cell line, a line obtained by selecting a line in which the neurons produce QRFP can be used. Whether neurons produce QRFP can be confirmed by a commonly used method using antibodies to QRFP.

### Hibernation Determining Method of the Present Invention

In the method for determining hibernation according to the present invention, the effect in a subject, of a drug that induces hibernation, a drug that is anticipated to induce hibernation, or a drug with the potential to induce hibernation is analyzed. If the subject has entered into a hibernation-like state, the hibernation-like state can be maintained or canceled. If the subject does not enter into a hibernation state, further treatment can be implemented or the treatment can be discontinued.

The method for determining hibernation according to the present invention may be a computational scientific method. The method for determining hibernation according to the present invention may not include medical intervention.

The method for determining hibernation according to the present invention is a method of determining (examining, predicting, estimating, computationally determining) whether a test compound induces hibernation in a mammal such as a human, or the potential for the test compound to induce hibernation in a mammal such as a human, the method including:

providing (or recording) an oxygen consumption amount and a core body temperature recorded under at least two different ambient environment temperature conditions for both before administration and after administration of the test compound in a mammal such as a human to which a test compound has been administered in regions of the anteroventral periventricular nucleus (AVPe), the medial preoptic area (MPA), and the periventricular nucleus (Pe);

estimating a correlation between the oxygen consumption amount and the core body temperature for each of before administration and after administration of the test compound; and

determining, based on the estimated correlation, whether an extent of decrease in the oxygen consumption amount when the core body temperature has decreased is reduced after administration in comparison to before administration, and determining whether an estimated value of the core body temperature when the oxygen consumption amount is assumed to be 0 is reduced after administration in comparison to before administration; wherein

the mammal is determined to hibernate if, after administration of the test compound, the extent of decrease in the oxygen consumption amount when the core body temperature has decreased is reduced in comparison to before administration, and the estimated value of the core body temperature when the oxygen consumption amount is assumed to be 0 is reduced after administration of the test compound in comparison to before administration. The mammal may be a non-human mammal.

The different ambient environment temperature conditions can be set using a temperature controller (e.g., the device of the first embodiment or the second embodiment). The oxygen consumption amount and core body temperature can be determined using an exhaled gas analyzer and a core body temperature gauge, respectively. As the exhaled gas analysis device and the core body temperature gauge, those provided in the device of the first embodiment or the second embodiment can be used.

### EXAMPLES

### Experimental Technique

### (1) Animals

All animal experiments were conducted at the International Institute for Integrative Sleep Medicine (IIIS) of the University of Tsukuba, and the RIKIN Center for Biosystems Dynamics Research (BDR) in accordance with guidelines for animal experiments. The approval of the animal experimentation committees of each institution was obtained, and thus NIH guidelines were followed. With the exception of the torpor-inducing experiment, mice were fed and watered freely and maintained at a T_{A} of 22° C., a relative humidity of 50% in a cycle of 12 h of lightness and 12 hours of darkness. It was confirmed that mice with a body weight of 34 g or heavier do not reproducibly exhibit FIT, and therefore heavy mice weighing 34 g or greater were excluded from the torpor experiments.

Qrfp-iCre mice were created through homologous recombination in C57BL/6N embryonic stem cells and transplantation in eight-cell stage embryos (ICR). A targeting vector was designed such that the entire coding region of the prepro-Qrfp sequence in exon 2 of the Qrfp gene is substituted with iCre and a pgk-Neo cassette, and so that the endogenous Qrfp promoter promotes the expression of iCre. Chimeric mice were crossbred with C57BL/6J females (Jackson Labs). The pgk-Neo cassette was deleted by crossbreeding with FLP66 mice backcrossed with C57BL/6J mice at least ten times. First, F1 hybrids were produced from heterozygotes crossbred with heterozygotes. These mice were backcrossed at least eight times with the C57BL/6J mice.

Rosa26^{dreaddm3} and Rosa26^{dreaddm4} mice were produced through homologous recombination in C57BL/6N embryonic stem cells, after which the same procedure as described above with regard to the Qrfp-iCre mice was implemented.

### (2) Viruses

As previously described³³, AAV was created using triple transfection and a helper-free method. The final purified virus was stored at −80° C. Titers of recombinant AAV vectors were measured by quantitative PCR. AAV₁₀-EF1α-DIO-TVA-mCherry, 4×10¹³; AAV₁₀-CAG-DIO-RG, 1×10¹³; AAV₁₀-EF1α-DIO-hM3Dq-mCherry, 1.64×10¹²; AAV₁₀-EF1α-DIO-mCherry, 1.44×10¹²; AAV₁₀-EF1α-DIO-SSFO-EYFP, 1.35×10¹²; AAV2/9-hsyn-DIO-TeTxLC-GFP, 6.24×10¹⁴; AAV2/9-hsyn-DIO-GFP, 4×10¹² genome copies/ml. Recombinant rabies vectors were created by a previously reported method^{22,34}. The SADΔG-GFP (EnvA) titer was 4.2×10⁸ infection units/ml.

### (3) Surgery

To inject the AAV vector, male Qrfp-iCre heterozygosity mice (8 to 12 weeks old) were anesthetized with isoflurane and placed in a stereotactic frame (David Kopf Instruments).

For chemogenetic manipulation, Qrfp-iCre mice were injected with AAV₁₀-EF1α-DIO-hM3Dq-mCherry at a rate of 0.1 μm/min using a Hamilton injection needle. The injection was made into the hypothalamus (for MB injection, anterior-posterior direction (AP), −0.46 mm; medial-lateral direction (ML), ±0.25 mm; dorsal ventral direction (DV), −5.75 mm; 0.50 μl at each site; LH injection; AP, −1.00 mm; ML, ±1.00 mm; DV, −5.00 mm, 0.30 μl at each site). The needle was left in place for 10 minutes after the injection.

For optogenetic manipulation, AAV10-EF1α-DIO-SSFO-EYFP was injected into one side in the AVPe (AP, 0.38 mm; ML, 0.25 mm; DV, −5.50 mm from the bregma). Next, optical fibers were implanted bilaterally above the AVPe (AP: 0.38 mm, ML: ±0.25 mm, DV: −5.20 mm), bilaterally at the DMH (AP: −1.70 mm, ML: ±0.25 mm, DV: −4.75 mm) or unilaterally at the RPa (AP: −6.00 mm, ML: 0.00 mm, DV: −5.50 mm) (FIG. 2j). After the injections, the mice were allowed to recover for a period of at least two weeks in individual cages, after which the mice were subjected to infrared thermal imaging experiments. Behavioral data was included only if these viruses were specifically targeted for Q-neurons and the optical fiber implants were precisely placed.

### (4) Recording Biological Signals

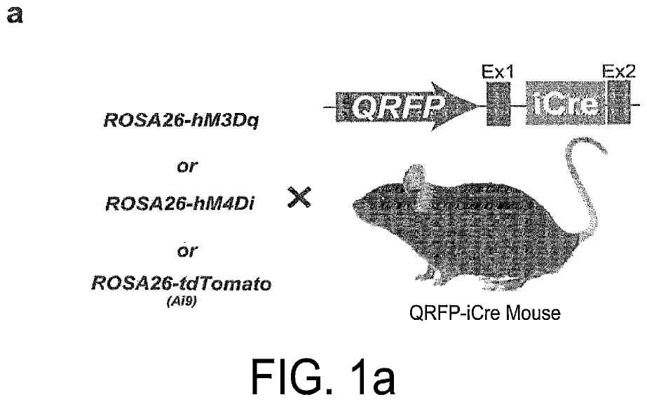

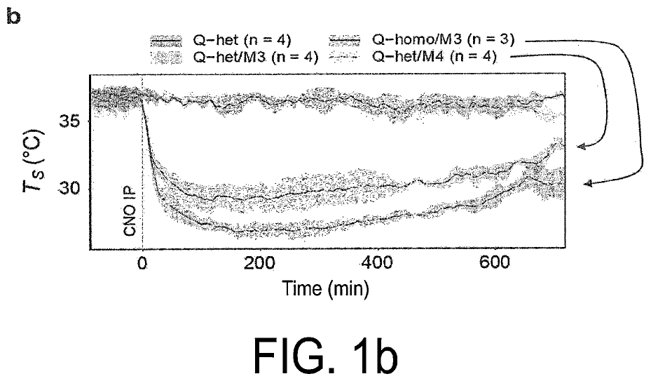

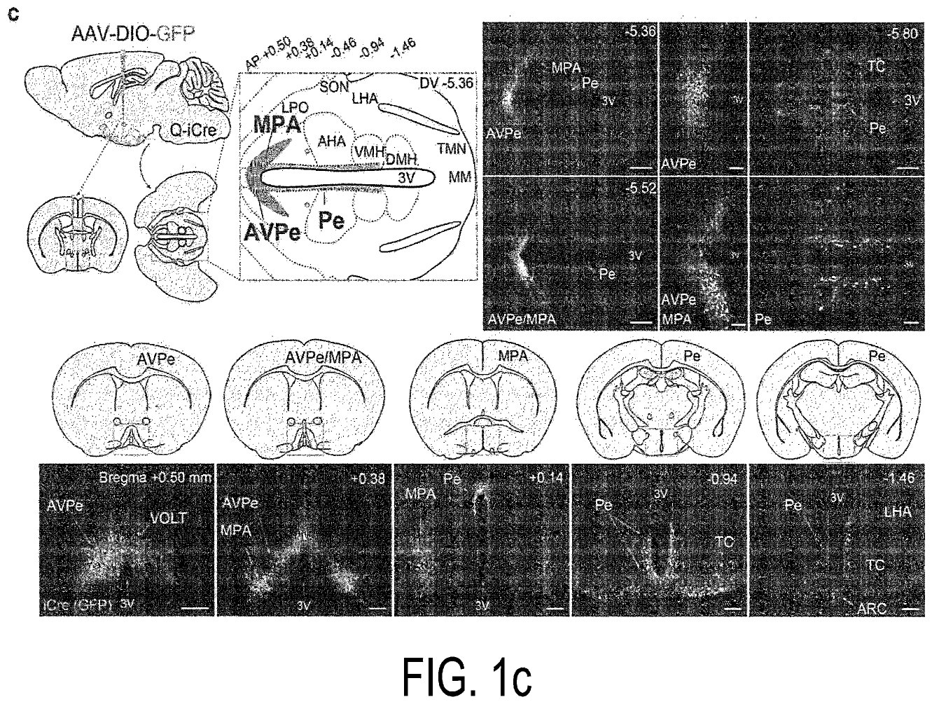

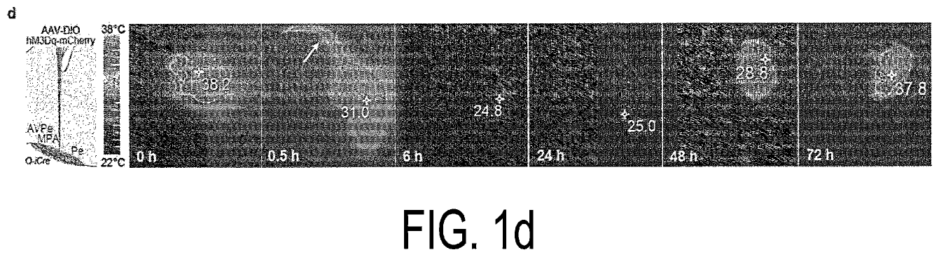

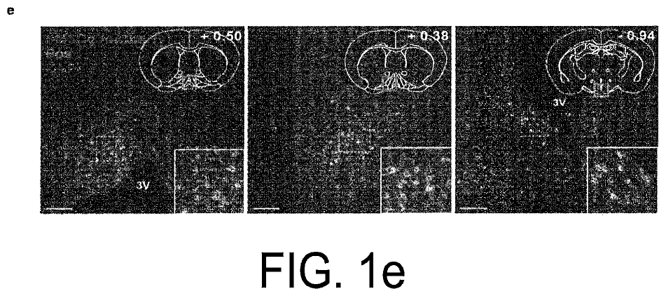

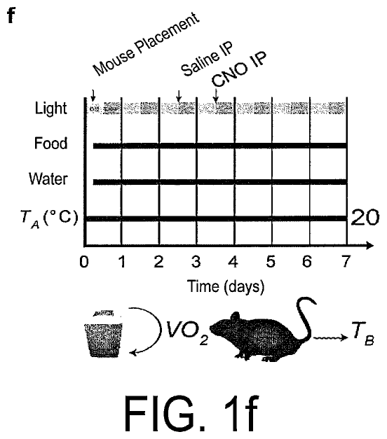

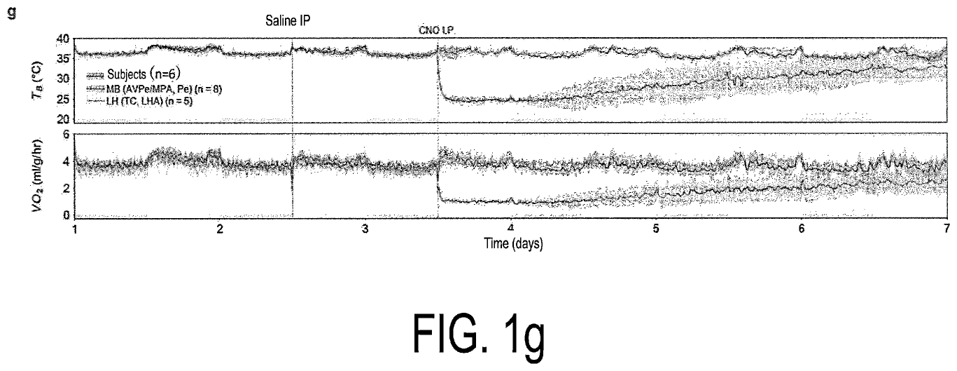

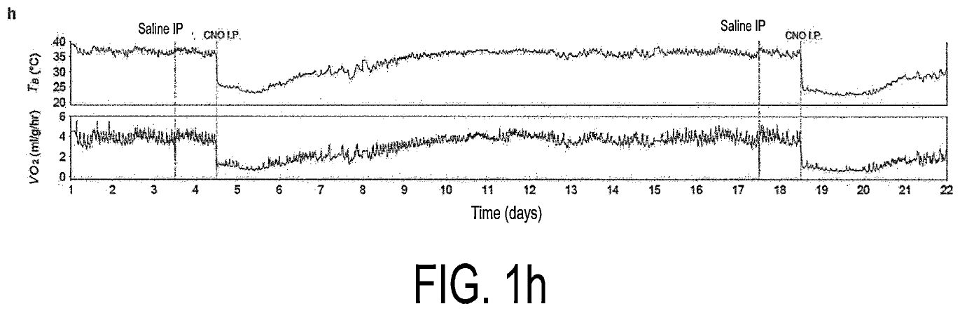

For thermographic analysis, the mice were inserted into an experiment cage (25×15×10 cm) and monitored using an infrared thermal imaging camera (InfReC R500 EX; Nippon Avionics) placed 30 cm above the cage floor. To clearly detect the surface temperature, the back hair was removed using hair clippers one day prior to the start of the experiment. Thermograms of DREADD and optogenetic experiments were collected at 0.5 Hz and 1 Hz, respectively, and analyzed using the InfReC Analyzer NS9500 Professional software (Nippon Avionics). The highest temperature of one frame was used as the T_{S} of the animal (FIG. 1d).

In order to record the core body temperature, oxygen consumption amount, EEG, ECG, and respiratory patterns, each animal was housed in a temperature controlled chamber (HC-100, Shin Factory or LP-400P-AR, Nippon Medical & Chemical Instruments Co., Ltd.). In order to continuously record the T_{B} (intraperitoneal temperature), a telemetry temperature sensor (TA11TA-F10, DSI) was implanted into the animal's abdominal cavity under general inhalation anesthesia at least 7 days prior to the recording. The VO 2 and carbon dioxide discharge rate (VCO₂) of the animal were continuously recorded using a respiratory gas analyzer (ARCO-2000 mass spectrometer, ARCO System). The respiratory quotient was calculated from the ratio of VCO₂/VO₂.

The EEG and ECG were recorded using a implanted remote measurement transmitter (F20-EET or HD-X02, DSI). For EEG recording, two stainless steel screws (1 mm in diameter) were soldered to a wire of a telemetry transmitter and inserted under general anesthesia through the skull of the cortex (AP, 1.00 mm; right, 1.50 mm from bregma or lambda). Two other wires from the transmitter were placed on the surface of the thoractic cavity, and an ECG was recorded. The mice were allowed to recover from surgery for at least 10 days. An EEG/ECG data collecting system consisted of transmitters, an analog-digital converter and a recording computer with the software Ponemah Physiology Platform (version 6.30, DSI). The sampling rate was 500 Hz for both the EEG and the ECG, and the data was converted into ASCII format for review. The heart rate was evaluated through a visual inspection of the waveform.

The respiratory flow was recorded using a non-invasive respiratory flow recording system³⁵. Specifically, a mouse was placed in metabolic chamber (TMC-1213-PMMA, Minamiderika Shokai) having an air flow of at least 0.3 L/min. The chamber was connected to a pressure sensor (PMD-8203-3G, Biotex), and the pressure difference between the outside and inside of the chamber was detected. If the animal is breathing, the pressure difference from the outside to the inside increases during inhalation and decreases during exhalation³⁵. An analog signal output from the sensor was converted to a digital signal by an AD converter (NI-9205, National Instruments) at 250 Hz, and stored in a computer using data logging software developed by Biotex Inc.

### (5) FIT Induction

An experiment for inducing daily torpor was designed to record the metabolism of the animal for at least three days. The animal was introduced into the chamber the day before recording started (day 0). The animal was allowed to freely consume food and water. T_{A} was set as indicated on day 0 and maintained at a constant level throughout the experiment. Power to the telemetry temperature sensor implanted in the mouse was turned on before the mouse was placed in the chamber. The standard experimental design was as follows. On day 2, the food was removed at ZT-0 in order to induce daily torpor. After 24 hours, the food was returned to each animal at ZT-0 of day 3.

### (6) Recording Metabolism During Drug Administration

The Dreadd agonist CNO (clozapine N-oxide, Abcam, ab141704) was dissolved in physiological saline at a dose of 100 μg/mL, and the solution was frozen at −20° C. The CNO solution was thawed on site and then administered to each mouse intraperitoneally at a dose of 1 mg/kg. The adenosineAl receptor agonist CHA (N⁶-cyclohexyl adenosine, Sigma-Aldrich, C9901) was dissolved in physiological saline at a concentration of 250 μg/mL, and the solution was administered intraperitoneally to each mouse at a dose of 2.5 mg/kg.

### (7) Recording Metabolism During General Anesthesia

In addition to the above-described above T_{B}, VO₂ and video recordings (refer to “Recording biological signals”), the entrance to the metabolic chamber was connected directly to the exit of an inhalation anesthesia device (NARCOBIT-E, Natsume Seisakusho Co., Ltd.). 1% isoflurane was supplied for 30 minutes at T_{A}=28° C., and followed by 90 minutes at T_{A}=12° C. After the experiment, the animals were warmed on a hot plate, and the recovery was confirmed.

### (8) Immunohistochemical Staining

Each mouse was deeply anesthetized with isoflurane, transcardially perfused with 10% sucrose in water, and then perfused with ice-cooled 4% paraformaldehyde (4% PFA) in a 0.1 M phosphate buffer having a pH of 7.4, and the brain was removed. After being left in the 4% PFA overnight at 4° C., the brain was fixed, incubated overnight at 4° C. in a 0.1 M phosphate buffered saline (PBS) having a pH of 7.4, in 30% sucrose, immersed in a Tissuue-Tek O.C.T. compound (Sakura) in a cryomold, and frozen at −80° C. until sectioned. With the use of a cryostat (CM1860, Leica), the brain was sliced into coronal sections in four equal series every 50 μm, collected in a 6-well plate filled with ice-cooled PBS, and washed three times with PBS at room temperature (RT). Unless otherwise specified, the following incubation process was carried out while gently shaking on an orbital shaker. Brain sections were incubated for 1 hour at room temperature in 1% Triton X-100 in PBS. Without shaking, the sections were blocked at room temperature for 1 hour using 10% Blocking One (NACALAI TESQUE) in 0.3% Triton X-100-treated PBS (blocking solution). The sections were incubated overnight at 4° C. in primary antibodies diluted with a blocking solution (the diluted solution and type of each antibody are as indicated below), and then washed three times, incubated overnight at 4° C. along with a secondary antibody, washed with PBS, and then mounted and covered with a cover glass using the HardSet Antifade Mating Medium (VECTASHIELD) containing DAPI.

The primary antibodies used in this research were rabbit anti-cFos (1:4000, ABE457, Millipore), goat anti-mCherry (1:15000, AB0040-200, SICGEN), rat anti-GFP (1:5000, 04404-84, NACALAI TESQUE), mouse anti-TH (1:1000, sc-25269, Santa Cruz Biotechnology), mouse anti-orexin A (1:200, sc-80263, Santa Cruz Biotechnology), and rabbit anti-MCH (1:2000, M8440, SIGMA). The secondary antibodies were as follows. Alexa Fluor 488 donkey anti-rat, 488 donkey anti-rabbit, 594-donkey anti-rabbit, 594 donkey anti-goat, 647 donkey anti-mouse, and 647 donkey anti-rabbit (1:1000, Invitrogen). For Nissl staining, sections were counter-stained in the secondary antibody process using NeuroTrace 435/455 Blue Flue Fluorescent Nissl Stain (1:500, N-21479, Invitrogen), and then covered with a cover glass using the FluorSave Reagent (Millipore). Brain regions were determined using a mouse brain map from Paxinos and Franklin³⁶.

### (9) In Situ Hybridization

Fluorescence in situ hybridization was implemented using the RNAscope Fluorescent Multiple Kit (Advanced Cell Diagnostics) and a probe designed for RNAscope Fluorescent Multiplex in situ hybridization (ACDBio RNAscope Probe-Mm-Qrfp #4643411, mCherry #43201, Mm-Slc32a1 #319191, mM-Slc17a6 #319171). The brain was dissected and immediately frozen in 2-methylbutane on dry ice and stored in a frozen-embedding medium at −80° C. Prior to dissection, the brain was cooled to −16° C. in a cryostat for 1 hour. A cryostat (Leica CM1860UV) was used to cut the brain into 20 μm coronal sections, and the sections were mounted on Superfrost Plus Microscope Slides (Fisherbrand). A pre-treatment method and an RNAscope Fluorescent Multiplex Assay were implemented precisely in accordance with the RNAscope Assay Guide (document numbers 320513 and 320293, respectively).

### (10) Retrograde Tracing of Q-Neurons

Male Qrfp-iCre mice (10 to 12 weeks old) were injected with the following viruses. AAV₁₀-DIO-TVA-mCherry and AAV₁₀-DIO-RG were delivered to express TVA-mCherry and RG in Q-neurons in the MB region (see the description above regarding the procedure and coordinates). After two weeks, SADΔG-GFP (EnvA) was injected into the same site. The starter neurons and input (single GFP positive) neurons were detected in whole brain sections using the Leica TCS SP8 laser confocal microscope and the Zeiss Axio Zoom. V16, respectively.

### (11) Blood Chemical Analysis

Blood was collected from the mice under anesthesia through a left ventricular puncture using a 25 gauge needle. The collected blood was stored on ice for less than 2 hours. The sample was centrifuged at 2000 G for 10 minutes at 4° C., and the supernatant was collected and frozen at −30° C. The frozen serum specimens were sent to FUJIFILM Wako Pure Chemical Corporation, and the concentrations of Na (mEq/L), K (mEq/L), Cl (mEq/L), AST (IU/L), ALT (IU/L) LDH (IU/L), CK (IU/L), GLU (mg/dL), and total ketone bodies (μmol/L) were measured.

### (12) Electrophysiological Analysis

The mice were decapitated under deep anesthesia using isoflurane (Pfizer). The brains were extracted and cooled in an ice-cold cutting solution containing the following (mM): 125 mM choline chloride, 25 mM NaHCO₃, 10 mM D(+)-glucose, 7 mM MgCl₂, 2.5 mM KCl, 1.25 mM NaH₂PO₄, and 0.5 mM CaCl₂) bubbled with O₂ (95%) and CO₂ (5%). Horizontal brain slices (thickness of 250 μm) including the hypothalamus were prepared using a vibratome (VT1200S, Leica) and were maintained at room temperature for one hour in artificial CSF (ACSF) containing the following (mM): 125 mM NaCl, 26 mM NaHCO₃, 10 mM D(+)-glucose, 2.5 mM KCl, 2 mM CaCl₂), and 1 mM MgSO₄ bubbled with O₂ (95%) and CO₂ (5%). The electrodes (5 to 8 MΩ) were filled with an internal solution containing the following (mM): 125 mM K-gluconate, 10 mM HEPES, 10 mM phosphocreatine, 0.05 mM tolbutamide, 4 mM NaCl, 4 mM ATP, 2 mM MgCl₂, 0.4 mM GTP, and 0.2 mM EGTA, with a pH of 7.3 and adjusted with KOH). Firing of hM3Dq-mCherry expressing neurons was recorded in a current-clamp mode at a temperature of 30° C. CNO (1 μM) was bath-applied, and the effect was examined. The membrane voltage and data acquisition were controlled using a combination of the MultiClamp 700B amplifier, the Digidata 1440A A/D converter and Clampex 10.3 software (Molecular Devices).

### (13) 3D Imaging of Transparent Mouse Brain

Transparent mouse brains were produced through the Sca/eS method as previously described³⁷. A ScaleS solution was produced using urea crystals (Wako Pure Chemical Industries, Ltd., 217-00615), D(−)-sorbitol (Wako Pure Chemical Industries, Ltd., 199-14731), methyl-β-cyclodextrin (Tokyo Chemical Industry Co., Ltd., M1356), γ-cyclodextrin (Wako Pure Chemical Industries, Ltd., 037-10643), N-acetyl-L-hydroxyproline (Skin Essential Active, Taiwan), dimethyl sulfoxide (DMSO) (Wako Pure Chemical Industries, Ltd., 043-07216), glycerol (Sigma, G9012), and Triton X-100 (Nacalai Tesque, 35501-15). The brains of the Qrfp-iCre mice injected with AAV-DIO-GFP were fixed and made transparent with ScaleS. Images were then acquired using a laser confocal microscope (Olympus, XLSLPN25XGMP (NA 1.00, WD: 8 mm) RI: 1.41 to 1.52).

### (14) Statistical Analysis

In this study, Bayesian statistics were applied to evaluate the hypothesis of the inventors and the experimental results. The inventors designed a statistical model with parameters representing the framework of the hypothesis and fit the model to the experimental results. Bayesian inference is used to estimate a posterior probability distribution of model parameters from a likelihood distribution and a prior probability distribution of the parameters. The post-distribution provides information about how the model can explain the hypothesis from the experimental results. The Bayesian model can explicitly include all types of uncertainties, and thus the Bayesian model can handle data with noise in an observation, or can utilize information from a small number of samples potentially having a wide range of uncertainty. Furthermore, the Bayesian model can use a hierarchical model to handle multiple layers of a plurality of groups with different numbers of samples. These advantages of Bayesian inference are all suitable for addressing problems often found in animal experiments. Model fitting was implemented using Hamiltonian Monte Carlo with a No-U-Turn Sampler as executed with version 2.18.0 of Stan with the RStan library³⁸ of version 3.52 of R³⁹. Convergence was evaluated by examining the trace plots:

### [Math. 1]

Gelman-Rubin \hat{R}

and also by estimating the number of effective samples. The prior probability density function of the model was defined to be weakly informative and conservative, and was stipulated in the following sections. Basic principles and techniques for the design of the statistical model were based on the book Statistical Rethinking⁴⁰. The model and data source code used in the analysis can both be obtained from https://briefcase.riken.jp/public/JjtgwAnqQ81AgyI. (Because of evaluation, the model and data source code are protected by the password “qih”, and are slated to be made available to the public.)

The body weights of the Qrfp-iCre mice were modeled through a state-space hierarchical model (code folder QRFP_KO_BW) at a predetermined age and lineage. Animals in each group (Qrfp-iCre mice of the wild type (n=9), the heterozygous type (n=9), and the homozygous type (n=10)) were raised in respective cages without identification of individual mice. When the unobservable baseline of body weight is defined as a time variable B_{t,s}, where t is the time point, and the lineage index (1, 2, and 3 for the wild type, the heterozygous type, and the homozygous type Qrfp-iCre mice, respectively) is expressed by the trend n_{t,s} and the total time point T, an observed state Y_{t,i} can be described by modeling observational error through a log-normal distribution as follows.

$$\begin{matrix}[ Math . 2 ] & \\ {Y}_{t , s} ∼ Log - normal ( log ( {B}_{t , s} ) - \frac{{( {σ}_{1} )}^{2}}{2} , {σ}_{1} ) & ( 1 )\end{matrix}$$

$$\{ \begin{matrix}{B}_{1 , s} = + {η}_{1} , t = 1 \\ {B}_{2 , s} - {B}_{1 , s} = {B}_{1 , s} + {η}_{2 , s} , t = 2 \\ {B}_{t , s} - {B}_{t - 1 , s} = {B}_{t - 1 , s} - {B}_{t - 2 , s} + {η}_{t , s} , t \geq 3\end{matrix} \qquad ( 2 )$$

$${η}_{t , s} ∼ Normal ( 0 , {σ}_{2} ) \qquad ( 3 )$$

$$t = 1 … T \qquad ( 4 )$$

$$s = \{ 1 , 2 , 3 \} \qquad ( 5 )$$

A uniform prior probability density function was applied to all parameters except σ1 and σ2, which were drawn from the standard half-normal distribution.

The spike frequency of Qrfp positive neurons in brain slices was modeled by creating parameters of the difference in spike frequency when neurons were activated by CNO (code folder Patch M3_CNO). When the total number of slices is K, and the observed spike frequencies of the control and the CNO-administered recording of the “i” th slice are denoted by Bᵢ and Cᵢ, respectively, Bᵢ is modeled by β_{BASE} with an observational error, and Cᵢ is modeled by the sum of β_{CNO} and β_{BASE} with the observational error. Because the spiking frequency is a positive real number, the error can be modeled by a log-normal distribution, and thus Bᵢ and Cᵢ can be described as follows.

$$\begin{matrix}[ Math . 3 ] & \\ {B}_{i} ∼ Log - normal ( log ( {β}_{BASE} ) - \frac{{( {σ}_{ERROR} )}^{2}}{2} , {σ}_{ERROR} ) & ( 6 )\end{matrix}$$

$${C}_{i} ∼ Log - normal ( log ( {β}_{BASE} + {β}_{CNO} ) - \frac{{( {σ}_{ERROR} )}^{2}}{2} , {σ}_{ERROR} ) \qquad ( 7 )$$

$${β}_{BASE} ∼ Normal ( 0 , {σ}_{BASE} ) \qquad ( 8 )$$

$${β}_{CNO} ∼ Normal ( 0 , {σ}_{CNO} ) \qquad ( 9 )$$

$$i = 1 … K \qquad ( 10 )$$

All σs were sampled from a standard half-normal distribution.

The T_{S} of optogenetically stimulated animals was modeled with a hierarchical multilayer model (FIG. 21, code folder SSFO_Opto). Animals of four groups were included in this experiment. T_{S} was recorded at 1 Hz, the median was saved every 10 seconds, and further analysis was implemented. All T_{S} recorded at 115 to 125 minutes after the first light stimulus were included in the analysis. When K is the total number of animals and Y is the T_{S} during a time of interest of a mouse j belonging to i, Y can be expressed as a sum of a global average parameter β, a group parameter β_{GROUP}, and an individual mouse parameter β_{MOUSE}, in association with observational noise modeled by a Cauchy distribution of a scale parameter σ_{ERROR}.

[Math. 4]

Y_{i,j}˜Cauchy(β+β_{GROUP[i]}+β_{MOUSE[j]},σ_{ERROR}) (11)

β_{GROUP}˜Normal(0,σ_{GROUP}) (12)

β_{MOUSE}˜Normal(0,σ_{MOUSE}) (13)

i={1,2,3,4} (14)

j=1 . . . K (15)

All as were sampled from a standard semi-normal distribution. The differences in T_{S} between each group were compared by estimating the average T_{S} of each group from the post-distribution, which is the sum of β and β_{GROUP} having normal distribution noise at a standard deviation of σ_{MOUSE}.

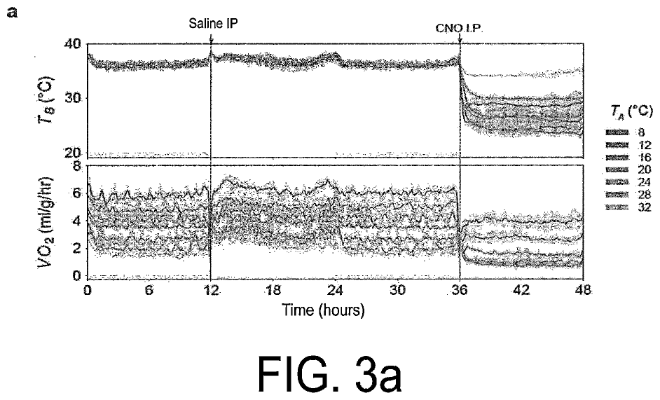

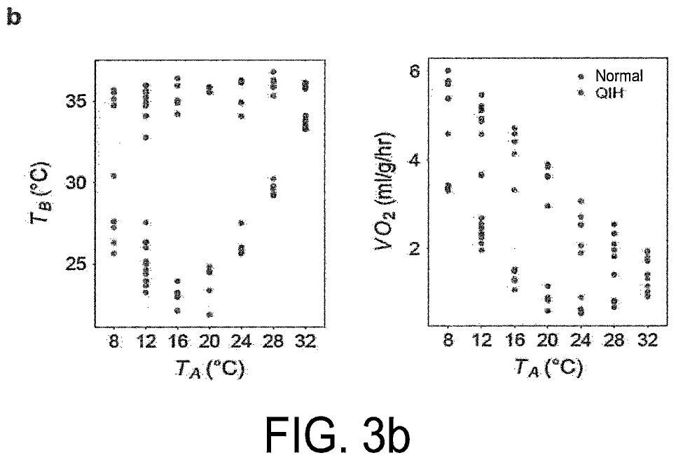

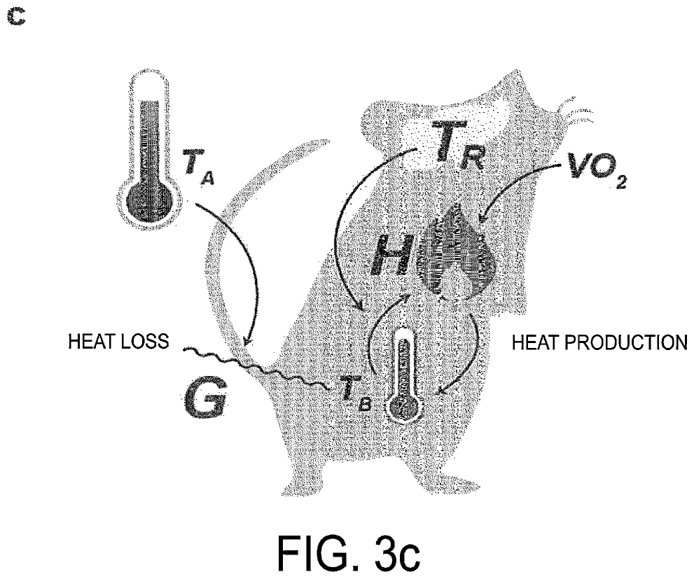

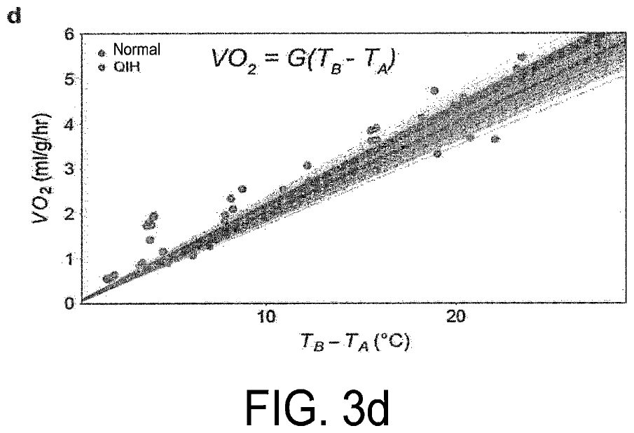

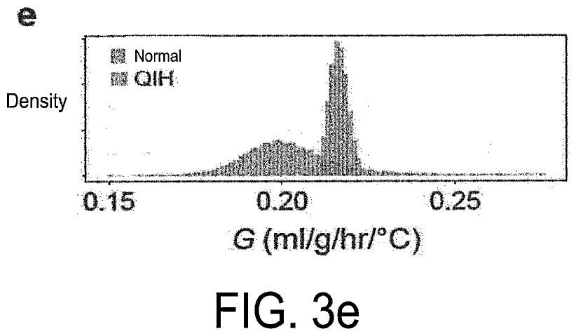

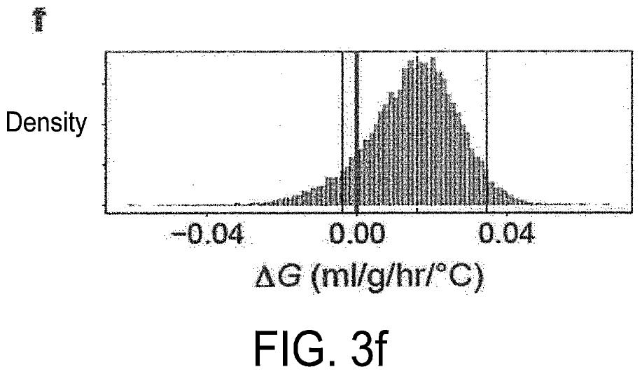

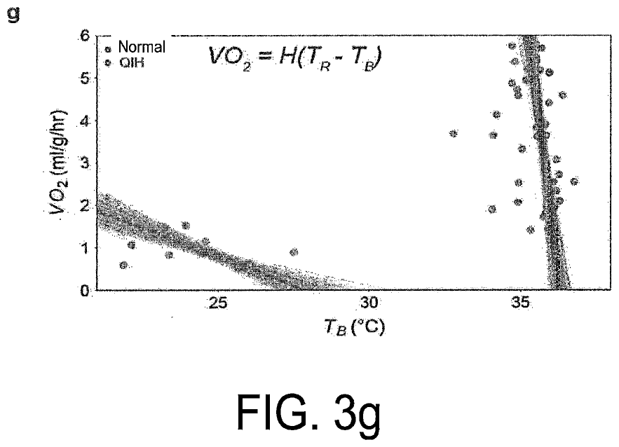

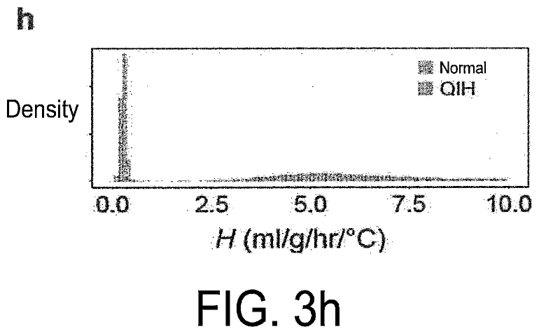

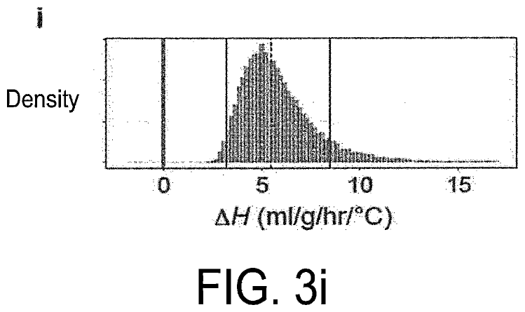

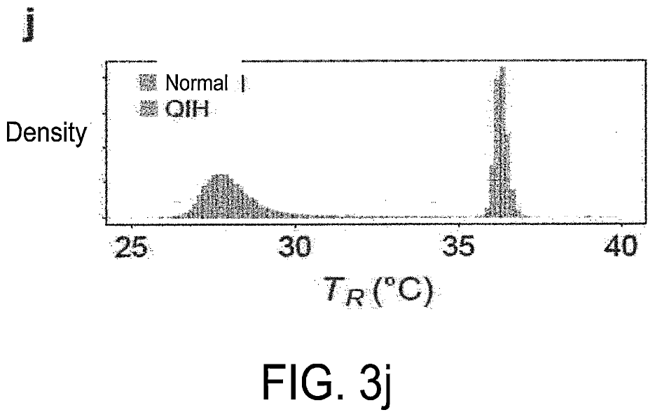

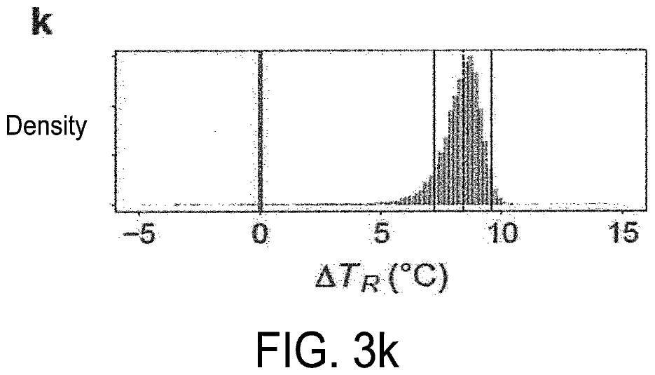

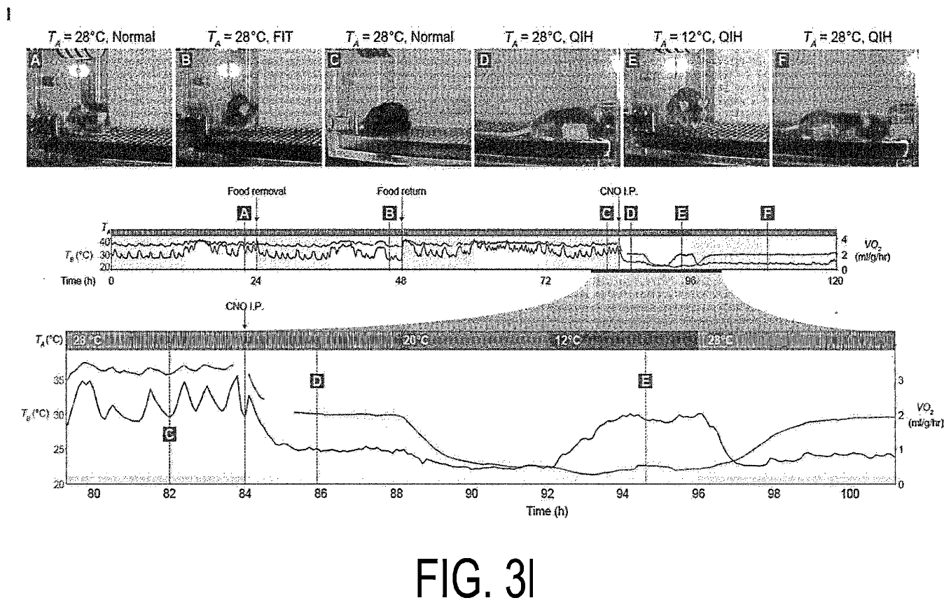

To evaluate the thermoregulatory system under QIH and normal conditions, the heat loss and heat production of the animals were described in a hierarchical multilayer model (FIG. 3c to FIG. 3k, code folder QIH_GTRH). Three parameters of G, T_{R} and H under two metabolic conditions, that is, normal and QIH conditions, were estimated from a metabolically stable state of the animals at various T_{A}s. The detailed method has been previously described³. In summary, a linear model formed from the controllable parameter T_{A} and the observable parameters T_{B} and VO₂ was fitted to the experimental results for both T_{B} and VO₂ using T_{A} as a predictor having normal distribution noise. Next, G, T_{R}, and H were estimated using the post-distribution of the slope and intercept coefficients for each model. In this analysis, the prior probability density function of the standard deviation of noise was a standard half-normal distribution, while the other parameters used a positive region of a uniform distribution except for the intercept coefficient of T_{B}, which used a uniform distribution attributed to negative values.

Circadian transition of metabolism in the Q-TeTxLC mice were analyzed by modeling the metabolism by clustering the recorded values in the L-phase and D-phase (code folder TeTxLC LD). In particular, when Y is the observed T_{B} of group i in phase j, Y can be expressed as a sum of the basal metabolism (L-phase metabolism) and difference of the D-phase, and the observational noise of the normal distribution becomes as follows.

$$\begin{matrix}[ Math . 5 ] & \\ {Y}_{i , j} ∼ Normal ( {β}_{BASE [ i ]} + {β}_{DARK [ i ]} {P}_{j} , {σ}_{ERROR} ) & ( 16 )\end{matrix}$$

$${β}_{BASE} ∼ Normal ( 0 , {σ}_{BASE} ) \qquad ( 17 )$$

$${β}_{DARK} ∼ Normal ( 0 , {σ}_{DARK} ) \qquad ( 18 )$$

$$i = \{ 1 : control , 2 : TeTxLC \} \qquad ( 19 )$$

$$j = \{ 1 : L - phase , 2 : D - phase \} \qquad ( 20 )$$

$$\{ \begin{matrix}{P}_{1} = 0 \\ {P}_{2} = 1\end{matrix} \qquad ( 21 )$$

All as were sampled from a standard semi-normal distribution. In the modeling of VO₂, since VO₂ assumes only a positive real number, the basic model structure was the same as that of T_{B} modeling, except that the observational error was modeled as a log-normal distribution.

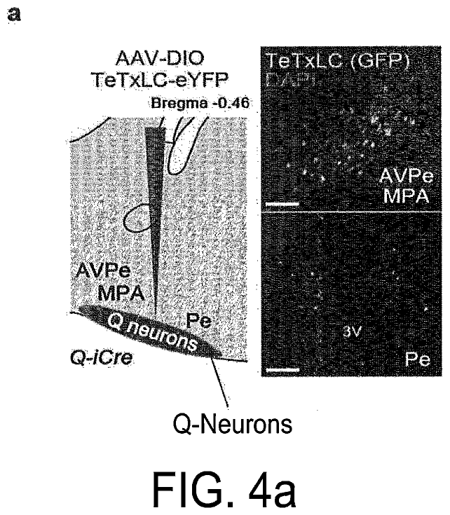

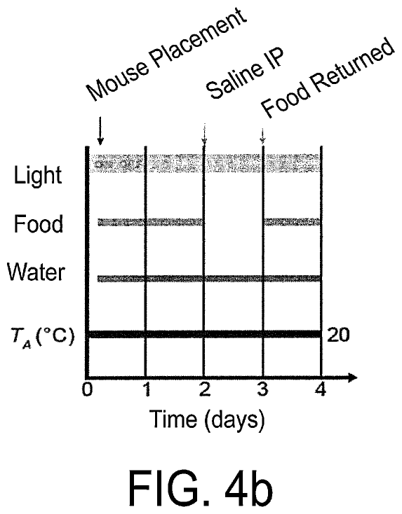

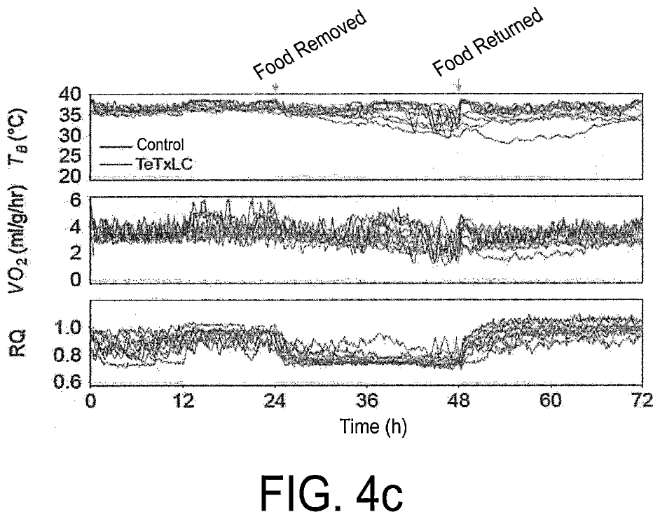

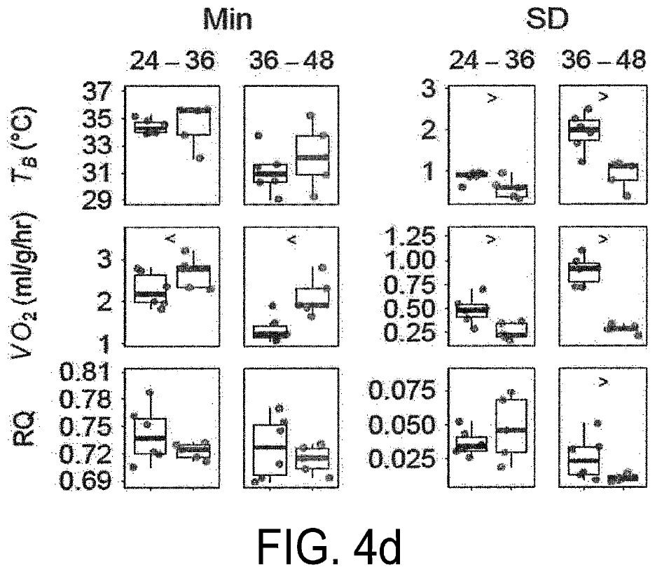

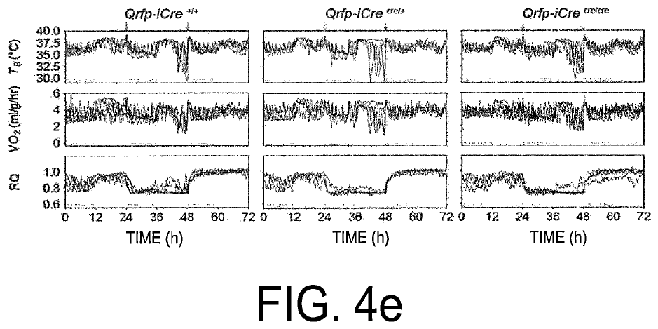

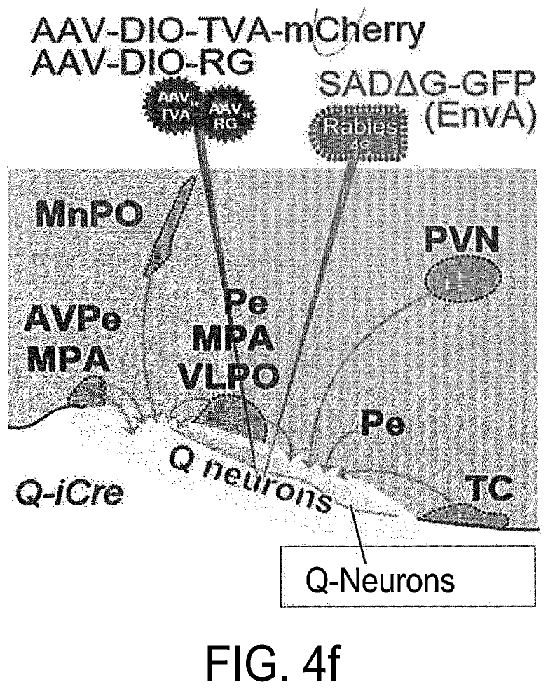

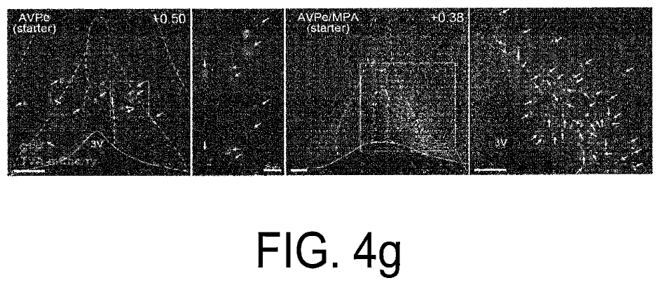

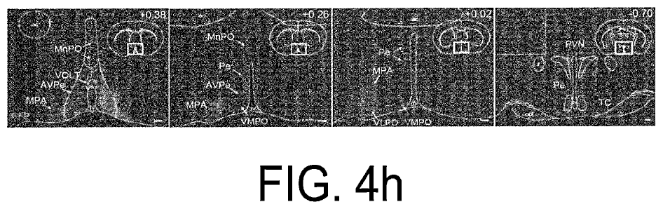

Metabolism during FIT in Q-TeTxLC mice was modeled with a hierarchical multilayer model (FIG. 4d, code folder TeTxLC FIT). The minimum Y value of group i in section j can be expressed as the sum of the average metabolism of group β_{0[i]} and the difference parameter β_{1[i,j]}.

[Math. 6]

Y_{i,j}·Normal(β_{0[i]}+β_{1[i,j]},σ_{ERROR}) (22)

β₀˜Normal(0,σ₀) (23)

β₁˜Normal(0,σ₁) (24)

i={1: 24 to 36 h,2: 36 to 48 h} (25)

j={1: Control,2: Q−TeTxLC} (26)

The identity of the mice was included as a predictor of observed values Yin order to model metabolic dispersion. In this manner, Y of a predetermined group of a certain section was modeled as a normal distribution, and this normal distribution used a mouse-dependent average α_{MOUSE} and a group and section-dependent parameter β_{GROUP,SECTION} as averages, and σ_{GROUP,SECTION} as the standard deviation.

[Math. 7]

Y_{i,j,k}˜Normal(αₖ+β_{i,j},σ_{i,j}) (27)

αₖ˜Normal(0,σ_{α}) (28)

β_{i,j}˜Normal(0,σ_{β}) (29)

σ_{i,j}˜Normal(0,σ_{σ}) (30)

i={1:Control,2:Q−TeTxLC} (31)

j={1:24-36 h,2:36-48 h} (32)

All as of equations (22) to (24) and (28) to (30) were sampled from a standard half-normal distribution. These models were used in the modeling of T_{B}, VO₂, and RQ. Even Y of these models can theoretically accept a negative real number, and in this case, good convergence occurred. Therefore, this model was also applied to VO₂ and RQ.

### Experiment and Results

### Example 1: Induction of Hypometabolism by Chemically Defined Hypothalamic Neuron Population

The pyroglutamylated RFamide peptide (QRFP), a hypothalamic neuropeptide, was discovered through a bioinformatics approach originally targeting the discovery of a new RFamide peptide^{9,10}. Qrfp peptide was also identified and purified from rat brains as an endogenous ligand of an orphan G-protein-coupled receptor hGPR103¹¹. The prepro-Qrfp mRNA localizes exclusively in the hypothalamus and is distributed in the periventricular nucleus (Pe), the lateral hypothalamus (LHA), and the tuber cinereum (TC)¹¹. Qrfp has been found to be involved in food intake, sympathetic regulation, and anxiety^{11,12}. The inventors created mice (Qrfp-iCre mice) having codon-improved Cre recombinase (iCre) knocked-in into the Qrfp gene. In order to obtain mice (Qrfp-iCre; Rosa26^{dreaddm3} mice) in which hM3Dq-mCherry is expressed exclusively in iCre-expressing neurons, the inventors crossbred CAG-hM3Dq-mCherry with Rosa26^{ddreadm3} mice having an upstream floxed transcription arrest element inserted at the Rosa26 gene locus. During excitatory chemogenetic experiments using Qrfp-iCre; Rosa26^{dreaddm3} mice, these mice exhibited a significant decline in motor activity and ultimately entered a sustained state of serious immobility beginning approximately 30 minutes after intraperitoneal (IP) injection of clozapine-N-oxide (CNO). It was noticed that the posture of these mice was similar to the observed posture during daily torpor, and therefore it was initially hypothesized that activation of iCre-positive cells in Qrfp-iCre mice induces daily torpor and is characterized by immobility and low T_{B} (as described below, it is clear that the hypothermia induced here is not daily torpor, but is a hibernation-like state). To evaluate this hypothesis, the surface body temperature (T_{S}) was measured using a thermography camera, and it was discovered that the CNO-induced immobile state of the Qrfp-iCre; Rosa26^{dreaddm3} mice was accompanied by significantly sustained hypothermia (FIG. 1B). The reduction of T_{S} began approximately 5 minutes after CNO administration and continued for approximately 12 hours. Subsequently, the mice spontaneously recovered from the hypothermic state without external re-heating.

In contrast, inhibitory DREADD manipulation through activation of hM4Di in iCre-positive neurons of Qrfp-iCre; Rosa26^{dreaddm4} mice did not have any effect on T_{S} (FIG. 1B). Importantly, hM3Dq-mediated activation of iCre-positive neurons in Qrfp-iCre; Rosa26^{dreadm3} mice induced significant hypothermia, even in homozygous Qrfp-iCre mice in which the prepro-Qrfp sequences were completely substituted by iCre in both alleles (FIG. 1B). This suggests that the Qrfp peptide itself is not required for inducing hypothermia. Rather, the degree of hypothermia is more pronounced in Qrfp knockout (Qrf-iCre homozygote) mice, suggesting the possibility that endogenous Qrfp itself counteracts hypothermia. This concurs with a previous observation of the inventors. Namely, when Qrfp is administered centrally, sympathetic nerve outflow increases, and the heart rate and blood pressure increase¹¹.

Therefore, Qrfp was identified as a chemical marker for hypothermia-inducing neurons. Next, although iCre-positive neurons are observed exclusively in the hypothalamus, they are distributed in several discrete hypothalamic regions of Qrfp-iCre mice, and therefore an attempt was made to identify hypothalamus regions that induce hypothermia. A Cre-activating AAV vector having a flip-excision (FLEX) switch¹³ was injected into the hypothalamus of the Qrfp-iCre mice using two different stereotaxic coordinates, namely mediobasal (MB) injection or lateral (LH) injection (see the methods section), and thereby iCre-positive neurons of inner and outer regions of the hypothalamus were separately manipulated. Through MB injection of the Cre-dependent AAV vector, it was possible to express specific genes of the iCre-positive neurons in the inner regions of the hypothalamus, namely, the anteroventral periventricular nucleus (AVPe), the medial preoptic area (MPA) and the Pe. However, those genes could not be expressed in the LHA (FIG. 1c). Through multi-color fluorescence in situ hybridization analysis, it was confirmed that most mCherry-positive cells in these regions express the Qrfp mRNA. After hM3Dq was expressed in this region by MB injection of AAV₁₀-EF1α-DIO-hM3Dq-mCherry into the Qrfp-iCre mice, an electrophysiological study using hypothalamus slices prepared from these mice was conducted, and it was confirmed that bath-application of the CNO strongly excited the mCherry-positive neurons. It was also found that IP injection of CNO into these mice causes a deeper, longer sustained hypothermia than the significant immobile state observed in the Qrfp-iCre; Rosa26^{dreaddm3} mice (FIG. 1B, FIG. 1d). An extremely low T_{B} state (below 30° C.) continued for 48 hours or longer (FIG. 1d). Through immunostaining with anti-Fos and anti-mCherry antibodies, numerous mCherry and Fos double positive neurons became clear in the AVPe, MPA and Pe regions, and the in vivo excitation of these neurons by CNO was confirmed (FIG. 1e).

From these observations, it was concluded that the iCre-positive neurons (these neurons are described below as quiescence-inducing neurons or Q neurons) in the AVPe/MPA and Pe regions of the Qrfp-iCre mice are mainly responsible for the induced hypothermic state. In the following experiments, unless otherwise stated, the inventors basically used Qrfp-iCre mice that were MB injected with AAV₁₀-EF1a-DIO-hM3Dq-mCherry (referred to as Q-hM3D mice) to induce hypothermia.

To further analyze the induced hypothermic state, a telemetry temperature sensor was implanted into the abdominal cavity of the Q-hM3D mice, and metabolism was continuously analyzed by respiratory gas analysis (FIG. 1f). The present study confirmed that the CNO-induced hypothermic state in the Q-hM3D mice is accompanied by a significant reduction in the O₂ consumption rate (VO₂: oxygen consumption amount) (FIG. 1g), and that T_{B} decreases concurrently with Tₛ after CNO administration. In contrast, excitatory DREADD manipulation of iCre-positive neurons in outer regions of the hypothalamus (LHA and TC) of Qrfp-iCre mice through LH injection of AAV₁₀-EF1a-DIO-hM3Dq-mCherry did not induce hypothermia (FIG. 1g).

During a Q-neuron-induced hypothermia/hypometabolism (QIH) state, the heart rate significantly decreased (from 758 beats/min two hours prior to CNO injection to 215 beats/min two hours after CNO injection) (average of n=3). The respiratory rate was reduced from 333 breaths/minute to an undetectable state (the ventilation amount of one time was less than the detection limit). At these timings, VO₂ was reduced from 3.60 to 1.17 ml/g/hr. In QIH, the mice exhibited a very low amplitude electroencephalogram (EEG), and this EEG clearly differed from those observed in non-rapid eye movement sleep characterized by high-amplitude slow waves. Blood serum chemical data suggests that blood glucose levels are reduced in QIH, which is presumably due to reduced gluconeogenesis due to low sympathetic tone. These observations further suggest that many body functions robustly decrease along with the reduction in T_{B} and VO₂ during QIH.

DREADD-mediated effects usually continue for only a few hours after CNO injection, but the DREADD-induced QIH in the Q-hM3D mice was sustained for a very long period of time. Surprisingly, at T_{A}=20° C., QIH with a T_{B} of less than 30° C. was sustained for 48 hours or longer with only one dose (1 mg/kg) of CNO, and it took about 1 week for the VO₂ to fully return to normal (FIG. 1h). After recovery from QIH, the mice were healthy and seemed to behave normally. QUI was reproducible in the same mice after repeated CNO injections, indicating the reversibility of this operation (FIG. 1h).

### Example 2: Induction of QIH by Action of Q-Neurons on Dorsomedial Hypothalamus

The axonal projection of Q-neurons was analyzed to clarify the mechanism for inducing QIH. Qrfp-iCre mice were injected with AAV₁₀-EF1a-DIO-GFP to express GFP specifically in Q-neurons (FIGS. 2a, 2b), after which GFP-positive fibers were observed in regions (regions pertaining to body temperature regulation and sympathetic nerve control) including the MPA, VOLT, paraventricular nucleus (PVN), the supraoptic nucleus (SON), the dorsomedial hypothalamus (DMH), the LHA, the tuberomammillary nucleus (TMN), the medial mammillary nucleus (MM), the periaqueductal gray (PAG), the lateral parabrachial nucleus (LPB), the locus ceruleus (LLC), the rostral ventrolateral medulla (RVLM), and the raphe pallidus nucleus (RPa) (FIG. 2c)¹⁴. The inventors found that the DMH received particularly abundant projections. The position of Q-neurons and projections to the DMH were further suggested from an analysis of the brain that was clarified with the ScaleS method (FIG. 2d).

Next, whether these Q-neurons are inhibitory or excitatory was confirmed using triple-color in situ hybridization of the Q-neurons. After it was confirmed that the CNO injection effectively induces QIH in Q-hM3D mice, these mice were subjected to in situ hybridization histochemical examinations. A transcription product that encodes for mCherry and probes that encode for vesicular glutamate transporter 2 (Vglut 2) and vesicular GABA transporter (Vgat), which is an excitatory and inhibitory marker, respectively, were used. The inventors also discovered that approximately two-thirds of the Q-neurons were i-positive and approximately two-fifths were Vglut2-positive (FIGS. 2e to 2i).

Among the regions containing abundant projections by Q-neurons (FIG. 2c), the inventors focused attention on the DMH. This is because thermogenesis-promoting neurons were previously identified in the DMH¹⁵. An optogenetic approach was used to clarify the function of axonal projections of Q-neurons into the DMH. Stabilized step function opsin (SSFO)¹⁶ was expressed by Q-neurons by injecting Qrfp-iCre mice (Q-SSFO mice) with AAV₁₀-DIO-SSFO-eYFP (FIG. 2j). The expression of SSFO in the AVPe, MPA and Pe regions was confirmed. In order to confirm the effect of optogenetic excitation on T_{S}, first, optical fibers were implanted in the AVPe/MPA where many cell bodies of Q-neurons are found (FIG. 2j), and then the mice were subjected to light-generating excitation of SSFO-positive cell bodies by applying a light pulse (a light pulse of a 1 second width). In this state, optogenetic excitation of the Q-neurons rapidly induced robust hypothermia that lasted for approximately 20 minutes (FIG. 2k). When the excitation of Q-neurons was repeated every 30 minutes for a total of four times, an even more profound hypothermic state occurred in association with a low T_{S} around the same level as the T_{A} (22° C.). Many Fos-positive neurons were identified in SSFO-eYFP-positive cells in the AVPe/MPA after excitation (FIG. 2j). Optogenetically-induced QIH clearly lasted for a shorter amount of time than QIH induced by hM3Dq-mediated pharmacogenetic excitation of Q-neurons (FIG. 2k). This suggests the possibility that Gq-mediated metabotropic signaling in Q-neurons that leads to changes in gene expression profiles plays a role in creating long-term sustainability of QIH.

Next, optical fibers were implanted into the DMH bilaterally to the Q-SSFO mice, and the optogenetic excitation was applied to the axonal fibers. This manipulation effectively decreased T_{S}, but was slightly weaker than induction by somal stimulation in the AVPe/MPA (FIGS. 2k, 2l). It is known that the RPa region contains sympathetic premotor neurons for thermogenesis via brown adipose tissue control¹⁷, and therefore as a control, the effect of the optogenetic excitation on Q-neuron fibers in the RPa was also examined. In addition, subtle effects of the optogenetic excitation of the Q-neuron fibers in the RPa on T_{S} were observed (FIGS. 2k, 2l). From these results, it is postulated that Q-neurons act mainly on the DMH, act to a smaller extent on the RPa, and induce QIH.

### Example 3: Decrease in Theoretical Set-Point Temperature During QIH

An increase in the temperature of the tails of the mice was observed immediately after QIH induction, and because QIH was induced by optogenetic or pharmacogenetic excitation of Q-neurons, this suggests that peripheral vasodilation occurs during the decreased T_{B}, and heat is released (FIG. 1d, FIG. 2k). Peripheral vasodilation without an increase in T_{B} suggests that the theoretical set-point of body temperature (T_{R}) was reset to a value lower than in the normal state, a feature that is observed in the hibernation state of hibernating animals. To evaluate this, a characteristic analysis of the thermoregulatory system of the mice during QIH was implemented. Heat conductance (G), H, and T_{R} can be estimated from T_{B} and VO₂ at multiple ambient temperatures (T_{A}) under conditions in which the animals are not engaged in external work and have a stable metabolism³. Q-hM3D mice were prepared, and the T_{B} and VO₂ were recorded during QIH at various TAS (8, 12, 16, 20, 24, 28 and 32° C.) (FIG. 3a). The average T_{B} and VO₂ eleven hours after IP injection of physiological saline or CNO were compared. During QIH, the animals were compared to a corresponding control, and at every T_{A}, the mice exhibited low T_{B} and VO₂ (FIG. 3b). When the heat-producing system is functioning properly, that is, when T_{R} is higher than T_{B}, and the thermoregulatory system increases VO₂ in an attempt to reach T_{R} (FIG. 3c), as T_{A} increases, T_{B} is increased and VO₂ is decreased. T_{B} and VO₂ exhibited minimum values at respectively different T_{A}, and a coordinated heat-producing property was exhibited only in a T_{A} range of from 16 to 24° C. (FIG. 3b). Therefore, further analysis was conducted using metabolic data at T_{A}=16, 20, and 24° C. during QIH. First, G was estimated from the relationship of T_{B}-T_{A} and VO₂ (FIG. 3d). Under normal and QIH conditions, the 89% highest posterior density interval (HPDI) of G was [0.212, 0.221] ml/g/hr/° C. and [0.182, 0.220] ml/g/hr/° C., respectively (FIG. 3e, hereafter, the 89% HPDI is indicated by two numbers in square brackets). Quantitatively, the posterior distribution (ΔG) of the difference in both G was [−0.0040, 0.0348] ml/g/hr/° C. (FIG. 3f) and included 0, suggesting that G under normal conditions and G under QIH conditions is indistinguishable. This differed from daily torpor, during which a lower G was exhibited than under normal conditions³. Second, H and T_{R} were estimated from T_{B} and VO₂ (FIG. 3g). H was [3.43, 8.72] ml/g/hr/° C. in the normal state and was [0.181, 0.369] ml/g/hr/° C. in QIH (FIG. 3h), indicating a 95.3% reduction in each median. The post-distribution (ΔH) of differences was [3.17, 8.48] ml/g/hr/° C. (FIG. 3i), which was positive, suggesting that the probability that these conditions differs is 89% or higher. This decrease in H was similar to the decrease in H during fasting-induced daily torpor (FIT)³. In particular, T_{R} was estimated to be [36.04, 36.60]° C. in the normal state, and [26.83, 29.13]° C. in QIH (FIG. 3j). The difference in T_{R} at each median was 8.41° C., the posterior distribution of the difference (ΔT_{R}) was [7.18, 9.57]° C., and thus a decrease in T_{R} during QIH was clearly demonstrated (FIG. 3k). In consideration of the very small shift³ in the theoretical set-point temperature in FIT, this observation emphasizes the similarity between QIH and hibernation, as well as the difference between QIH and daily torpor.

To provide more evidence of the reduction in T_{R} during QIH, which is a prominent feature of hibernation, the relationship between the posture and metabolism of each mouse was observed when the T_{A} was dynamically changed during QIH (n=4, representative data of one mouse in FIG. 3l, and data of other three mice). The very stable, long-term hypometabolic state of QIH such as in a hibernating animal made it possible to examine this in mice. FIT was induced in Q-hM3D mice at a setting of T_{A}=28° C. (A and B in FIG. 3l). After a recovery period of 24 hours, QIH was induced by administering CNO (C, D, E, and F in FIG. 3l). Interestingly, at T_{A}=28° C., the mice exhibited an extended posture during QIH, and this posture is normally seen in animals exposed to high temperature environments (D in FIG. 3l). This clearly differed from the typical sitting posture that was observed during FIT at T_{A}=28° C. (B in FIG. 3l). This behavior observation further demonstrates that T_{R} is lower in QIH than in the FIT or normal state. Moreover, when T_{A} was lowered to 12° C., the mouse returned to a sitting position (E in FIG. 3l) similar to daily torpor. These results strongly support the idea that during QIH, T_{R} decreases, but bodily functions and behavior are still regulated to adapt to changes in T_{A}.

It is well known that during hibernation, the metabolic rate of animals is low, but the metabolism is actively regulated in response to the T_{A}. Similarly, in QIH, animals exposed to a T_{A} of 16° C. or lower exhibited significantly larger VO₂ compared to animals exposed to a T_{A} of 20° C. or 28° C. (FIG. 3b). In fact, this is similar to a prior report¹⁸ pertaining to hibernating animals that exhibit a metabolic increase when T_{A} is lowered to a certain level. This feature of active hypometabolism during QIH was also confirmed in individual animals (FIG. 3l). The behavior and metabolic response of mice in QIH were entirely different than those observed in the normal state in which the bodily functions attempt to maintain T_{B} in a narrow range.

In order to compare metabolic function during QIH to an anesthetized state, the inventors recorded the metabolic transition during general anesthesia under a plurality of TAs. As expected, animals under anesthesia did not exhibit an increase in VO₂ or a change in posture even when exposed to a low T_{A}. The metabolic state induced by systemic delivery of the adenosine AJAR agonist (6)N-cyclohexyl-adenosine (CHA), which is used to induce a hypothermic state, was also examined¹⁹. IP injection of CHA (2.5 mg/kg) into wild-type mice effectively induced a hypothermic/hypometabolic state, but the mice did not react to a low T_{A} (12° C.) through either behavior (posture and shivering) or an increase in VO₂. T_{B} at 20° tends to be higher in CHA-induced hypothermia than in QIH, but T_{B} and VO₂ were further reduced in CHA-induced hypothermia. On the other hand, when T_{A} was set to 12° C., these parameters increased in QIH (FIG. 3b). These observations indicate that QIH differs entirely from general anesthesia or CHA-induced passive hypothermia, and that a hypothermic state is induced by blocking the T_{B} regulatory system.

### Example 4: Q-Neurons Participate in Normal Fasting-Induced Daily Torpor

QIH is more similar to hibernation than daily torpor, but daily torpor may be thought to be a mild state of hibernation. Therefore, an examination was conducted to determine whether Q-neurons all participate in daily torpor. In addition, common or similar mechanisms may play a role in inducing hibernation and daily torpor²⁰. To investigate the role of Q-neurons in a daily torpor, Qrfp-iCre mice (Q-TeTxLC mice) were injected with AAV_{2/9}-hSyn-DIO-TeTxLC-eYFP to express a tetanus toxin light chain (TeTxLC) specifically in Q-neurons, and an examination was conducted to determine whether blocking of SNARE complex-mediated neurotransmission in Q-neurons affects FIT (FIG. 4b). Simultaneous infusion of AAV_{2/9}-hSyn-DIO-TeTxLC-eYFP and AAV₁₀-EF1α-DIO-hM3Dq-mCherry completely eliminated the effect of CNO on T_{B}, suggesting that blocking the SNARE complex eliminates the QIH-inducing capacity of Q-neurons. The inventors discovered that the normal architecture of FIT was disrupted in all of the Q-TeTxLC mice. Rapid oscillatory fluctuations in metabolism were not observed in these mice during fasting (FIGS. 4c, 4d). This suggests that the function of Q-neurons is necessary for inducing a rapid decrease in T_{B} during FIT. Interestingly, the gradual decrease in T_{B} observed in these mice means that a metabolism-reducing mechanism that is not dependent on Q-neurons is present during FIT. In addition, the Q-TeTxLC mice had fewer circadian fluctuations in T_{B} than the control mice, which suggests that Q-neurons have a primary role in circadian regulation of T_{B}. In particular, homozygous Qrfp-iCre mice lacking the QRFP peptide showed normal FIT (FIG. 4e). These observations suggest that Q-neurons are an essential constituent element for inducing daily torpor and play an important role in the rapid shift of body temperature in daily torpor, but QRFP does not.

In order to clarify the neuronal mechanism regulating the activity of Q-neurons, the inventors identified upstream neuronal populations that make direct synaptic contact with Q-neurons through recombinant pseudotyped rabies virus vector (SADΔG(EnvA))-mediated labeling²¹ (FIG. 4f). TVA-mCherry and rabies glycoprotein (RG) were expressed in Q-neurons using Cre-activating AAV vectors²² in Qrfp-iCre mice, after which SADΔG-GFP (EnvA) was injected into the same site. Starter cells that were double-positive for TVA-mCherry and GFP were found in the AVPe/MPA and Pe regions (FIG. 4g). Input neurons in which Q-neurons (GFP positive but mCherry negative) were directly synaptically input were identified in the median preoptic area (MnPO), the PVN and the MPA (FIG. 4h). The input neurons were also observed inside and around the AVPe/Pe regions, suggesting the presence of local interneurons that regulate the function of Q-neurons, and also the likelihood that Q-neurons constitute microcircuits with interneurons within the AVPe/MPA and Pe regions. These observations suggest that Q-neurons receive relatively sparse direct input from the intra-hypothalmic region. Since FIT is induced by fasting, Q-neurons are expected to monitor negative energy balance. Neurons of PVH have been shown to receive abundant input from ARC²³, and thus the input from PVH to Q-neurons may serve to convey information pertaining to the nutritional state. PVH input may also convey circadian information from the suprachiasmatic nucleus (SCN).

MPA participates in the regulation of T_{B}^{24,25}, and therefore reciprocal interaction between Q-neurons and the MPA may play an important role in body temperature regulation. Input neurons are also contained in the ventromedial preoptic area (VMPO). In previous studies, warmth-sensing neurons of the VMPO were identified as BDNF and PACAP double-positive neurons. Excitation of these cells also induced hypothermia²⁶. This effect is much smaller than that induced by the excitement of Q-neurons, but it is possible that functional interaction exists between VMPO^{PACAP/BDNF} neurons and Q-neurons. Also, DREADD excitation of TRPM2-positive cells in the POA was shown to induce hypothermia. TRPM2 is highly expressed ubiquitously in the AVPe/MPA and the POA including a region containing input neurons to Q-neurons, and therefore TRPM2-induced hypothermia can possibly be induced by direct and/or indirect activation of Q-neurons²⁷.

The Q-neurons are localized along the third ventricle (3V), and dendrites of these neurons extend along the ependyma of the 3V and regions near circumventricular organs (FIG. 1c) Therefore, these Q-neurons might also sense humoral factors that are released by tanycytes and ependymal cells, factors in the cerbrospinal fluid, or capillary vessels.

### Discussion

Here, the presence of a novel hypothalamic neuron population with specific chemical (=expression of Qrfp) and tissue-based (AVPe/MPA) characteristics in mice was demonstrated, and excitation of this population induces an active hypometabolism that is very similar to hibernation. QIH, which is this state, shares two primary properties with hibernation. The first is a decrease in T_{R}, and the second is actively regulated hypometabolism. During QIH, mice actively regulate bodily functions according to the external environment. Many other physiological parameters, such as decreased heart rate, weak respiration, and a low potential electroencephalogram, suggest a similarity between QIH and hibernation²⁹. Through an analysis of Fos expression, a previous study indicated that cells near the 3V are activated during hibernation in thirteen-lined ground squirrels³⁰. This activation pattern is very similar to the region in which Q-neurons are localized, suggesting that hibernation nerves might also use Q-neurons to induce hibernation.

It is very surprising that the mice were able to enter a hibernation-like state (=QIH). The ability to hibernate exists among distantly related mammals including rodents, caniformia, and even primates, and therefore it is reasonable to hypothesize that the neuronal mechanism of hibernation is preserved in a wide range of mammalian species, but these systems are not mobilized in non-hibernating animals in normal conditions. Since the Qrfp gene is preserved in also humans, it can be inferred that it may be possible to exhibit an active hypometabolic state when Q-neurons are excited. In this study, it was also confirmed that the DMH is a major effector site of Q-neurons. Future studies identifying QIH-induced neurons in the DMH will further clarify the mechanism of QIH. Q-neurons have the potential to also act on other regions identified in this study. For example, it was recently reported that SON plays an important role in general anesthesia and sleep³².

It was also found that Q-neurons are necessary for fasting-induced daily torpor in mice. However, the detection of an increase in Q-neuron activity during fasting of mice through Fos staining or fiber photometer (data not presented) could not be repeated, suggesting that the low level activation of Q-neurons may be sufficient for inducing hypothermia in daily torpor.

The induced hibernation in non-hibernating animals presented in this study is a promising advancement towards understanding the neuronal mechanism of active hypometabolism, and this study provides a method for examining how each tissue adopts a hibernation-like hypometabolic state. Furthermore, in association with the future development of a method for selectively exciting Q-neurons, QIH could provide a new approach for the development of a method that enables clinical application of synthetic hibernation in humans with significant benefits in medicine, including the potential to reduce systemic tissue damage after a heart attack or stroke, and use in the preservation of organs for transplant.

### REFERENCES

1. Bouma, H. R. et al. Induction of torpor: Mimicking natural metabolic suppression for biomedical applications. J. Cell. Physiol. 227, 12851290 (2012).
2. Geiser, F. Metabolic Rate and Body Temperature Reduction During Hibernation and Daily Torpor. Annu. Rev. Physiol. 66, 239274 (2004).
3. Sunagawa, G. A. & Takahashi, M. Hypometabolism during Daily Torpor in Mice is Dominated by Reduction in the Sensitivity of the Thermoregulatory System. Sci. Rep. 6, 37011 (2016).
4. Florant, G. L. & Heller, H. C. CNS regulation of body temperature in euthermic and hibernating marmots (Marmota flaviventris). Am. J. Physiol. 232, R203-8 (1977).
5. Heller, H. C. & Colliver, G. W. CNS regulation of body temperature during hibernation. Am. J. Physiol. 227, 5839 (1974).
6. Iliff, B. W. & Swoap, S. J. Central adenosine receptor signaling is necessary for daily torpor in mice. AJP Regul. Integr. Comp. Physiol. 303, R477R484 (2012).
7. Melvin, R. G. & Andrews, M. T. Torpor induction in mammals: recent discoveries fueling new ideas. Trends Endocrinol. Metab. 20, 490498 (2009).
8. Griko, Y. & Regan, M. D. Synthetic torpor: A method for safely and practically transporting experimental animals aboard spaceflight missions to deep space. Life Sci. Sp. Res. 16, 101107 (2018).
9. Fukusumi, S. et al. A New Peptidic Ligand and Its Receptor Regulating Adrenal Function in Rats. J. Biol. Chem. 278, 4638746395 (2003).
10. Chartrel, N. et al. Identification of 26RFa, a hypothalamic neuropeptide of the RFamide peptide family with orexigenic activity. Proc. Natl. Acad. Sci. 100, 1524715252 (2003).
11. Takayasu, S. et al. A neuropeptide ligand of the G protein-coupled receptor GPR103 regulates feeding, behavioral arousal, and blood pressure in mice. Proc Natl Acad Sci USA 103, 74387443 (2006).
12. Okamoto, K. et al. QRFP-Deficient Mice Are Hypophagic, Lean, Hypoactive and Exhibit Increased Anxiety-Like Behavior. PLoS One 11, e0164716 (2016).
13. Atasoy, D., Aponte, Y., Su, H. H. & Stemson, S. M. A FLEX switch targets Channelrhodopsin-2 to multiple cell types for imaging and long-range circuit mapping. J. Neurosci. 28, 702530 (2008).
14. Nakamura, K. Central circuitries for body temperature regulation and fever. Am. J. Physiol. Integr. Comp. Physiol. 301, R1207R1228 (2011).
15. Zhao, Z.-D. et al. A hypothalamic circuit that controls body temperature. Proc. Natl. Acad. Sci. U.S.A 114, 20422047 (2017).
16. Yizhar, O. et al. Neocortical excitation/inhibition balance in information processing and social dysfunction. Nature 477, 171 (2011).
17. Morrison, S. F. Central control of body temperature. F1000Research 5, (2016).
18. Ortmann, S. & Heldmaier, G. Regulation of body temperature and energy requirements of hibernating alpine marmots (Marmota marmota). Am. J. Physiol. Regul. Integr. Comp. Physiol. 278, R698-704 (2000).
19. Tupone, D., Madden, C. J. & Morrison, S. F. Central activation of the A1 adenosine receptor (A1AR) induces a hypothermic, torpor-like state in the rat. J. Neurosci. 33, 1451225 (2013).
20. Heldmaier, G., Ortmann, S. & Elvert, R. Natural hypometabolism during hibernation and daily torpor in mammals. Respir. Physiol. Neurobiol. 141, 317329 (2004).
21. Wickersham, I. R. et al. Monosynaptic Restriction of Transsynaptic Tracing from Single, Genetically Targeted Neurons. Neuron 53, 639647 (2007).
22. Saito, Y. C. et al. Monoamines Inhibit GABAergic Neurons in Ventrolateral Preoptic Area That Make Direct Synaptic Connections to Hypothalamic Arousal Neurons. J. Neurosci. 38, 63666378 (2018).
23. Andermann, M. L. & Lowell, B. B. Toward a Wiring Diagram Understanding of Appetite Control. Neuron 95, 757778 (2017).
24. Wang, T. A. et al. Thermoregulation via Temperature-Dependent PGD2 Production in Mouse Preoptic Area. Neuron 114 (2019). doi:10.1016/j.neuron.2019.04.035
25. Tan, C. L. & Knight, Z. A. Review Regulation of Body Temperature by the Nervous System. Neuron 98, 3148 (2018).
26. Tan, C. L. et al. Warm-Sensitive Neurons that Control Body Temperature. Cell 167, (2016).
27. Song, K. et al. The TRPM2 channel is a hypothalamic heat sensor that limits fever and can drive hypothermia. Science (80-.). 353, 13931398 (2016).
28. Oomura, Y. et al. A new brain glucosensor and its physiological significance. Am. J. Clin. Nutr. 55, 278S-282S (1992).
29. Walker, J. M., Glotzbach, S. F., Berger, R. J. & Heller, H. C. Sleep and hibernation in ground squirrels (Citellus spp):electrophysiological observations. Am. J. Physiol. Integr. Comp. Physiol. 233, R213R221 (1977).
30. Bratincsak, A. et al. Spatial and temporal activation of brain regions in hibernation: c-fos expression during the hibernation bout in thirteen-lined ground squirrel. J. Comp. Neurol. 505, 44358 (2007).
31. Dausmann, K. H., Glos, J., Ganzhorn, J. U. & Heldmaier, G. Hibernation in a tropical primate. Nature 429, 825826 (2004).
32. Jiang-Xie, L.-F. et al. A Common Neuroendocrine Substrate for Diverse General Anesthetics and Sleep. Neuron 113 (2019). doi:10.1016/j.neuron.2019.03.033
33. Mieda, M. et al. Cellular clocks in AVP neurons of the scn are critical for interneuronal coupling regulating circadian behavior rhythm. Neuron 85, 11031116 (2015).
34. Osakada, F. & Callaway, E. M. Design and generation of recombinant rabies virus vectors. Nat. Protoc. 8, 1583601 (2013).
35. Sunagawa, G. A. et al. Mammalian Reverse Genetics without Crossing Reveals Nr3a as a Short-Sleeper Gene. Cell Rep. 14, 662677 (2016).
36. Keith, B. J., Franklin & Paxinos, G. The mouse brain in stereotaxic coordinates. (Academic Press, 2007).
37. Hama, H. et al. ScaleS: an optical clearing palette for biological imaging. Nat. Neurosci. 18, 15181529 (2015).
38. Stan Development Team. RStan: the R interface to Stan. (2018).
39. R Core Team. R: A language and environment for statistical computing. (2017).
40. McElreath, R. Statistical Rethinking: A Bayesian Course with Examples in R and Stan. (CRC Press, 2016).

1. A device for stimulating, in a brain of a living subject, pyroglutamylated RFamide peptide (QRFP)-producing neurons in at least one region selected from the group consisting of an anteroventral periventricular nucleus (AVPe), a medial preoptic area (MPA), and a periventricular nucleus (Pe), the device comprising:
a controller configured to transmit a control signal controlling generation of a voltage;
a voltage generator configured to receive the control signal from the controller and generate a voltage;
a stimulation probe configured to be electrically connected proximally to the voltage generator and include an electrical stimulation electrode at a distal end, the stimulation probe having sufficient length for accessing QRFP-producing neurons from a brain surface and generating an electrical stimulus at the electrical stimulation electrode at the distal end through the voltage from the voltage generator;
an outside air temperature gauge;
a core body temperature gauge;
an exhaled gas analysis unit configured to measure an oxygen concentration in exhaled gas; and
a recording unit configured to record a measured outside air temperature and at least one numeric value selected from the group consisting of a core body temperature and an oxygen concentration.

2. A device for stimulating, in a brain of a living subject, pyroglutamylated RFamide peptide (QRFP)-producing neurons in at least one region selected from the group consisting of an anteroventral periventricular nucleus (AVPe), a medial preoptic area (MPA), and a periventricular nucleus (Pe), the device comprising:
a controller configured to transmit a control signal controlling a discharge of a QRFP-producing neuron stimulating compound;
a storage for the compound;
a compound transmitter configured to receive the control signal from the control unit and transmit the compound from the storage section of the compound;
a guide configured to include a compound discharge port and a flow path for the compound to the discharge port, and deliver the compound to QRFP-producing neurons;
an outside air temperature gauge;
a core body temperature gauge;
an exhaled gas analysis unit configured to measure an oxygen concentration in exhaled gas; and
a recording unit configured to record a measured outside air temperature and at least one numeric value selected from the group consisting of a core body temperature and an oxygen concentration.

3. The device according to claim 1, further comprising a determination unit configured to determine whether a subject is in a hypothermic state based on the outside air temperature and the core body temperature recorded in the recording unit.

4. The device according to claim 1, further comprising a determination unit configured to determine whether a subject is in a hypometabolic state based on the outside air temperature, the core body temperature, and the oxygen concentration recorded in the recording unit.

5. The device according to claim 1, further comprising a determination unit configured to determine whether a subject is in a hibernation-like state based on the outside air temperature, the core body temperature, and the oxygen concentration recorded in the recording unit.

6. The device according to claim 3, wherein the controller transmits a control signal for continuously or intermittently stimulating QRFP-producing neurons until the subject is determined to be in any one state selected from the group consisting of a hypothermic state, a hypometabolic state, and a hibernation-like state.

7. A method of reducing a theoretical set-point temperature of a body temperature in a mammalian subject, the method comprising applying an excitatory stimulus to pyroglutamylated RFamide peptide (QRFP)-producing neurons.

8. The method according to claim 7, wherein the QRFP-producing neurons are neurons in at least one region selected from the group consisting of an anteroventral periventricular nucleus (AVPe), a medial preoptic area (MPA), and a periventricular nucleus (Pe).

9. The method according to claim 7 or 8, wherein the excitatory stimulus is a stimulus selected from the group consisting of chemical stimuli, magnetic stimuli, and electrical stimuli.

10. A method of screening a substance applying an excitatory stimulus to pyroglutamylated RFamide peptide (QRFP)-producing neurons present in an anteroventral periventricular nucleus (AVPe), a medial preoptic area (MPA), and a periventricular nucleus (Pe) regions, the method comprising:
contacting a test compound and the QRFP-producing neurons with each other;
measuring an excitement of the QRFP-producing neurons; and
selecting a test compound that applies an excitatory stimulus to the QRFP-producing neurons.

11. A method of determining whether a test compound induces hibernation in a mammal, the method comprising:
providing an oxygen consumption amount and a core body temperature that are recorded under at least two different ambient environment temperature conditions for both before administration and after administration of the test compound in the mammal such as a human to which a test compound has been administered in regions including an anteroventral periventricular nucleus (AVPe), a medial preoptic area (MPA), and a periventricular nucleus;
estimating a correlation between the oxygen consumption amount and the core body temperature for each of before administration and after administration of the test compound; and
determining, based on the estimated correlation, whether an extent of decrease in the oxygen consumption amount when the core body temperature has decreased is reduced after administration in comparison to before administration, and determining whether an estimated value of the core body temperature when the oxygen consumption amount is assumed to be 0 is reduced after administration in comparison to before administration;
wherein the mammal is determined to hibernate if, after administration of the test compound, the extent of decrease in the oxygen consumption amount when the core body temperature has decreased is reduced in comparison to before administration, and the estimated value of the core body temperature when the oxygen consumption amount is assumed to be 0 is reduced after administration of the test compound in comparison to before administration.

12. A device for determining hibernation, the device comprising:
a recording unit configured to record, in a mammal such as a human to which a test compound has been administered in regions including an anteroventral periventricular nucleus (AVPe), a medial preoptic area (MPA), and a periventricular nucleus (Pe), record an oxygen consumption amount and a core body temperature recorded under at least two different ambient environment temperature conditions for both before administration and after administration of the test compound;
a calculation unit configured to estimate a correlation between the oxygen consumption amount and the core body temperature for each of before administration and after administration of the test compound, to determine, based on the estimated correlation, whether an extent of decrease in the oxygen consumption amount when the core body temperature has decreased is reduced after administration in comparison to before administration, and to determine whether an estimated value of the core body temperature when the oxygen consumption amount is assumed to be 0 is reduced after administration in comparison to before administration;
and a determination unit configured to determine that the mammal is hibernating if, after administration of the test compound, the extent of decrease in the oxygen consumption amount when the core body temperature has decreased is reduced in comparison to before administration, and the estimated value of the core body temperature when the oxygen consumption amount is assumed to be 0 is reduced after administration of the test compound in comparison to before administration.

13. The device according to claim 2, further comprising a determination unit configured to determine whether a subject is in a hypothermic state based on the outside air temperature and the core body temperature recorded in the recording unit.

14. The device according to claim 2, further comprising a determination unit configured to determine whether a subject is in a hypometabolic state based on the outside air temperature, the core body temperature, and the oxygen concentration recorded in the recording unit.

15. The device according to claim 2, further comprising a determination unit configured to determine whether a subject is in a hibernation-like state based on the outside air temperature, the core body temperature, and the oxygen concentration recorded in the recording unit.

16. The device according to claim 14, wherein the controller transmits a control signal for continuously or intermittently stimulating QRFP-producing neurons until the subject is determined to be in any one state selected from the group consisting of a hypothermic state, a hypometabolic state, and a hibernation-like state.
